SEQUENCE LISTING (168 sequences)

SEQ ID NO: 1 - DNA, 21 bp, Artificial Sequence
  CDS: part of the complementary determining region-3 (CDR3) in the V(14 family (BV14 gene) of T cell receptors (TCR) in patients with rheumatoid arthritis (RA)
agccaagctgacgggacccat

SEQ ID NO: 2 - DNA, 21 bp, Artificial Sequence
  CDS: part of the complementary determining region-3 (CDR3) in the V(16 family (BV16 gene) of TCR in patients with RA
agttccgggggcagtctgttc

SEQ ID NO: 3 - protein, 7 aa, Homo sapiens
  Peptide: conserved amino acid sequence derived from CDR3 of TCR beta-chain BV14 in patients with RA
SQADGTH

SEQ ID NO: 4 - protein, 7 aa, Homo sapiens
  Peptide: conserved amino acid sequence derived from CDR3 of TCR beta-chain BV16 in patients with RA
SSGGSLF

SEQ ID NO: 5 - protein, 4 aa, Homo sapiens
  Peptide: amino acid sequence motif derived from CDR3 of TCR beta-chain BV16 in patients with RA
SWGG

SEQ ID NO: 6 - protein, 113 aa, Homo sapiens
  Domain: amino acid sequence of human ( chain variable region V(14 of T cell receptors
MGPQLLGYVVLCLLGAGPLEAQVTQNPRYLITVTGKKLTVTCSQNMNHEYMSWYRQDPGLGLRQIYYSMNVEVTDKGDVPEGYKVSRKEKRNFPLILESPSPNQTSLYFCASS

SEQ ID NO: 7 - protein, 96 aa, Homo sapiens
  Domain: amino acid sequence of human ( chain variable region V(16 of T cell receptors
IEAGVTQFPSHSVIEKGQTVTLRCDPISGHDNLYWYRRVMGKEIKFLLHFVKESKQDESGMPNNRFLAERTGGTYSTLKVQPAELEDSGVYFCASS

SEQ ID NO: 8 - DNA, 21 bp, Artificial Sequence
  primer_bind: forward primer specific for TCR BV1 used in real-time PCR analysis
aagcacctgatcacagcaact

SEQ ID NO: 9 - DNA, 21 bp, Artificial Sequence
  primer_bind: reverse primer specific for TCR BV1 used in real-time PCR analysis
tagttcagagtgcaagtcagg

SEQ ID NO: 10 - DNA, 23 bp, Artificial Sequence
  primer_bind: forward primer specific for TCR BV2 used in real-time PCR analysis
ggttatctgtaagagtggaacct

SEQ ID NO: 11 - DNA, 21 bp, Artificial Sequence
  primer_bind: reverse primer specific for TCR BV2 used in real-time PCR analysis
aggatgggcactggtcactgt

SEQ ID NO: 12 - DNA, 24 bp, Artificial Sequence
  primer_bind: forward primer specific for TCR BV3 used in real-time PCR analysis
tcgagatatctagtcaaaaggacg

SEQ ID NO: 13 - DNA, 21 bp, Artificial Sequence
  primer_bind: reverse primer specific for TCR BV3 used in real-time PCR analysis
ggtgctggcggactccagaat

SEQ ID NO: 14 - DNA, 22 bp, Artificial Sequence
  primer_bind: forward primer specific for TCR BV4 used in real-time PCR analysis
aagcagggatatctgtcaacgt

SEQ ID NO: 15 - DNA, 21 bp, Artificial Sequence
  primer_bind: reverse primer specific for TCR BV4 used in real-time PCR analysis
ttcagggctcatgttgctcac

SEQ ID NO: 16 - DNA, 21 bp, Artificial Sequence
  primer_bind: forward primer specific for TCR BV5 used in real-time PCR analysis
gatcaaaacgagaggacagca

SEQ ID NO: 17 - DNA, 22 bp, Artificial Sequence
  primer_bind: reverse primer specific for TCR BV5 used in real-time PCR analysis
agcaccaaggcgctcacattca

SEQ ID NO: 18 - DNA, 21 bp, Artificial Sequence
  primer_bind: forward primer specific for TCR BV6 used in real-time PCR analysis
ctcaggtgtgatccaatttca

SEQ ID NO: 19 - DNA, 21 bp, Artificial Sequence
  primer_bind: reverse primer specific for TCR BV6 used in real-time PCR analysis
cccccgctctgtgcgctggat

SEQ ID NO: 20 - DNA, 25 bp, Artificial Sequence
  primer_bind: forward primer specific for TCR BV7 used in real-time PCR analysis
catgggaatgacaaataagaagtct

SEQ ID NO: 21 - DNA, 21 bp, Artificial Sequence
  primer_bind: reverse primer specific for TCR BV7 used in real-time PCR analysis
tggctgcagggcgtgtaggtg

SEQ ID NO: 22 - DNA, 21 bp, Artificial Sequence
  primer_bind: forward primer specific for TCR BV8 used in real-time PCR analysis
ccccgccatgaggtgacagag

SEQ ID NO: 23 - DNA, 21 bp, Artificial Sequence
  primer_bind: reverse primer specific for TCR BV8 used in real-time PCR analysis
gagtccctgggttctgagggc

SEQ ID NO: 24 - DNA, 21 bp, Artificial Sequence
  primer_bind: forward primer specific for TCR BV9 used in real-time PCR analysis
ccaaaatacctggtcacacag

SEQ ID NO: 25 - DNA, 22 bp, Artificial Sequence
  primer_bind: reverse primer specific for TCR BV9 used in real-time PCR analysis
ccagggaattgatgtgaagatt

SEQ ID NO: 26 - DNA, 22 bp, Artificial Sequence
  primer_bind: forward primer specific for TCR BV10 used in real-time PCR analysis
acctagacttctggtcaaagca

SEQ ID NO: 27 - DNA, 21 bp, Artificial Sequence
  primer_bind: reverse primer specific for TCR BV10 used in real-time PCR analysis
ggactggatctccaaggtaca

SEQ ID NO: 28 - DNA, 23 bp, Artificial Sequence
  primer_bind: forward primer specific for TCR BV11 used in real-time PCR analysis
ttatagggacaggaaagaagatc

SEQ ID NO: 29 - DNA, 21 bp, Artificial Sequence
  primer_bind: reverse primer specific for TCR BV11 used in real-time PCR analysis
atgtgagggcctggcagactc

SEQ ID NO: 30 - DNA, 23 bp, Artificial Sequence
  primer_bind: forward primer specific for TCR BV12 used in real-time PCR analysis
caagacacaagatcacagagaca

SEQ ID NO: 31 - DNA, 21 bp, Artificial Sequence
  primer_bind: reverse primer specific for TCR BV12 used in real-time PCR analysis
ggcagcagactccagagtgag

SEQ ID NO: 32 - DNA, 23 bp, Artificial Sequence
  primer_bind: forward primer specific for TCR BV13 used in real-time PCR analysis
tgaagacaggacagagcatgaca

SEQ ID NO: 33 - DNA, 21 bp, Artificial Sequence
  primer_bind: reverse primer specific for TCR BV13 used in real-time PCR analysis
cacagatgtctgggagggagc

SEQ ID NO: 34 - DNA, 23 bp, Artificial Sequence
  primer_bind: forward primer specific for TCR BV14 used in real-time PCR analysis
acccaagatacctcatcacagtg

SEQ ID NO: 35 - DNA, 21 bp, Artificial Sequence
  primer_bind: reverse primer specific for TCR BV14 used in real-time PCR analysis
agaggtctggttggggctggg

SEQ ID NO: 36 - DNA, 23 bp, Artificial Sequence
  primer_bind: forward primer specific for TCR BV15 used in real-time PCR analysis
tcacaaagacaggaaagaggatt

SEQ ID NO: 37 - DNA, 21 bp, Artificial Sequence
  primer_bind: reverse primer specific for TCR BV15 used in real-time PCR analysis
ggggatggcagactctaggga

SEQ ID NO: 38 - DNA, 22 bp, Artificial Sequence
  primer_bind: forward primer specific for TCR BV16 used in real-time PCR analysis
gttccccagccacagcgtaata

SEQ ID NO: 39 - DNA, 21 bp, Artificial Sequence
  primer_bind: reverse primer specific for TCR BV16 used in real-time PCR analysis
cagttctgcaggctgcacctt

SEQ ID NO: 40 - DNA, 22 bp, Artificial Sequence
  primer_bind: forward primer specific for TCR BV17 used in real-time PCR analysis
gtccccaaagtacctgttcaga

SEQ ID NO: 41 - DNA, 21 bp, Artificial Sequence
  primer_bind: reverse primer specific for TCR BV17 used in real-time PCR analysis
agctgtcgggttcttttgggc

SEQ ID NO: 42 - DNA, 21 bp, Artificial Sequence
  primer_bind: forward primer specific for TCR BV18 used in real-time PCR analysis
agacacctggtcaggaggagg

SEQ ID NO: 43 - DNA, 21 bp, Artificial Sequence
  primer_bind: reverse primer specific for TCR BV18 used in real-time PCR analysis
tgccgaatctcctcgcactac

SEQ ID NO: 44 - DNA, 24 bp, Artificial Sequence
  primer_bind: forward primer specific for TCR BV19 used in real-time PCR analysis
ccaggacatttggtcaaaggaaaa

SEQ ID NO: 45 - DNA, 21 bp, Artificial Sequence
  primer_bind: reverse primer specific for TCR BV19 used in real-time PCR analysis
cagtgccgtgtctcccggttc

SEQ ID NO: 46 - DNA, 19 bp, Artificial Sequence
  primer_bind: forward primer specific for TCR BV20 used in real-time PCR analysis
gaccctggtgcagcctgtg

SEQ ID NO: 47 - DNA, 21 bp, Artificial Sequence
  primer_bind: reverse primer specific for TCR BV20 used in real-time PCR analysis
gaggaggagcttcttagaact

SEQ ID NO: 48 - DNA, 24 bp, Artificial Sequence
  primer_bind: forward primer specific for TCR BV21 used in real-time PCR analysis
cccagatataagattacagagaaa

SEQ ID NO: 49 - DNA, 21 bp, Artificial Sequence
  primer_bind: reverse primer specific for TCR BV21 used in real-time PCR analysis
ctggatcttgagagtggagtc

SEQ ID NO: 50 - DNA, 23 bp, Artificial Sequence
  primer_bind: forward primer specific for TCR BV22 used in real-time PCR analysis
cacagatgggacaggaagtgatc

SEQ ID NO: 51 - DNA, 21 bp, Artificial Sequence
  primer_bind: reverse primer specific for TCR BV22 used in real-time PCR analysis
gtcctccagctttgtggaccg

SEQ ID NO: 52 - DNA, 21 bp, Artificial Sequence
  primer_bind: forward primer specific for TCR BV23 used in real-time PCR analysis
aagagggaaacagccactctg

SEQ ID NO: 53 - DNA, 21 bp, Artificial Sequence
  primer_bind: reverse primer specific for TCR BV23 used in real-time PCR analysis
cagctccaaggagctcatgtt

SEQ ID NO: 54 - DNA, 24 bp, Artificial Sequence
  primer_bind: forward primer specific for TCR BV24 used in real-time PCR analysis
ccaagataccaggttacccagttt

SEQ ID NO: 55 - DNA, 21 bp, Artificial Sequence
  primer_bind: reverse primer specific for TCR BV24 used in real-time PCR analysis
caggcctggtgagcggatgtc

SEQ ID NO: 56 - DNA, 22 bp, Artificial Sequence
  primer_bind: forward primer specific for TCR BV25 used in real-time PCR analysis
aaaacatcttgtcagaggggaa

SEQ ID NO: 57 - DNA, 21 bp, Artificial Sequence
  primer_bind: reverse primer specific for TCR BV25 used in real-time PCR analysis
tgaatcctcaagcttcgtagc

SEQ ID NO: 58 - DNA, 19 bp, Artificial Sequence
  primer_bind: forward primer specific for TCR BC used in real-time PCR analysis
cagcgcccttgtgttgatg

SEQ ID NO: 59 - DNA, 20 bp, Artificial Sequence
  primer_bind: reverse primer specific for TCR BC used in real-time PCR analysis
aagcgctggcaaaagaagaa

SEQ ID NO: 60 - DNA, 18 bp, Artificial Sequence
  primer_bind: BC primer used for run-off reactions
cgacctcgggtgggaaca

SEQ ID NO: 61 - DNA, 19 bp, Artificial Sequence
  primer_bind: FAM (expand)-labeled BC primer used for run-off reactions
cacagcgacctcgggtggg

SEQ ID NO: 62 - DNA, 21 bp, Artificial Sequence
  primer_bind: FAM (expand)-labeled BJ primer used for run-off reactions
actgtgagtctggtgccttgt

SEQ ID NO: 63 - DNA, 24 bp, Artificial Sequence
  primer_bind: FAM (expand)-labeled BJ primer used for run-off reactions
acaacggttaacttggtccccgaa

SEQ ID NO: 64 - DNA, 24 bp, Artificial Sequence
  primer_bind: FAM (expand)-labeled BJ primer used for run-off reactions
ggtcctctacaacagtgagccaac

SEQ ID NO: 65 - DNA, 24 bp, Artificial Sequence
  primer_bind: FAM (expand)-labeled BJ primer used for run-off reactions
aagagagagagctgggttccactg

SEQ ID NO: 66 - DNA, 19 bp, Artificial Sequence
  primer_bind: FAM (expand)-labeled BJ primer used for run-off reactions
ggagagtcgagttccatca

SEQ ID NO: 67 - DNA, 24 bp, Artificial Sequence
  primer_bind: FAM (expand)-labeled BJ primer used for run-off reactions
tgtcacagtgagcctggtcccatt

SEQ ID NO: 68 - DNA, 21 bp, Artificial Sequence
  primer_bind: FAM (expand)-labeled BJ primer used for run-off reactions
cctggcccgaagaactgctca

SEQ ID NO: 69 - DNA, 24 bp, Artificial Sequence
  primer_bind: FAM (expand)-labeled BJ primer used for run-off reactions
gtcctccagtacgctcagcctaga

SEQ ID NO: 70 - DNA, 21 bp, Artificial Sequence
  primer_bind: FAM (expand)-labeled BJ primer used for run-off reactions
tgcctgggccaaaatactgcg

SEQ ID NO: 71 - DNA, 21 bp, Artificial Sequence
  primer_bind: FAM (expand)-labeled BJ primer used for run-off reactions
tccccgcgccgaagtactgaa

SEQ ID NO: 72 - DNA, 18 bp, Artificial Sequence
  primer_bind: FAM (expand)-labeled BJ primer used for run-off reactions
tcgagcaccaggagccgc

SEQ ID NO: 73 - DNA, 21 bp, Artificial Sequence
  primer_bind: FAM (expand)-labeled BJ primer used for run-off reactions
ctgctgccggccccgaaagtc

SEQ ID NO: 74 - DNA, 21 bp, Artificial Sequence
  primer_bind: FAM (expand)-labeled BJ primer used for run-off reactions
tgaccgtgagcctggtgcccg

SEQ ID NO: 75 - protein, 20 aa, Homo sapiens
  Domain: CDR3 amino acid sequence of BV16 clonotype derived from ST specimen of RA patients
YFCASSQDSGGGGEQFFGPG

SEQ ID NO: 76 - DNA, 60 bp, Artificial Sequence
  CDS: CDR3 nucleic acid sequence of BV16 clonotype derived from ST specimen of RA patients
tatttctgtgccagcagccaagatagcggggggggaggtgagcagttcttcgggccagga

SEQ ID NO: 77 - protein, 20 aa, Homo sapiens
  Domain: CDR3 amino acid sequence of BV16 clonotype derived from ST specimen of RA patients
YFCASSRLGQGYNEQFFGPG

SEQ ID NO: 78 - DNA, 60 bp, Artificial Sequence
  CDS: CDR3 nucleic acid sequence of BV16 clonotype derived from ST specimen of RA patients
tatttctgtgccagcagccgactgggacagggctacaatgagcagttcttcgggccagga

SEQ ID NO: 79 - protein, 20 aa, Homo sapiens
  Domain: CDR3 amino acid sequence of BV16 clonotype derived from ST specimen of RA patient
YFCASSQDLDSYNEQFFGPG

SEQ ID NO: 80 - DNA, 60 bp, Artificial Sequence
  CDS: CDR3 nucleic acid sequence of BV16 clonotype derived from ST specimen of RA patients
tatttctgtgccagcagccaagatctggacagctacaatgagcagttcttcgggccagga

SEQ ID NO: 81 - protein, 20 aa, Homo sapiens
  Domain: CDR3 amino acid sequence of BV16 clonotype derived from ST specimen of RA patient
YFCASSQGTSGITEQFFGPG

SEQ ID NO: 82 - DNA, 60 bp, Artificial Sequence
  CDS: CDR3 nucleic acid sequence of BV16 clonotype derived from ST specimen of RA patients
tatttctgtgccagcagccaggggactagcgggatcactgagcagttcttcgggccagga

SEQ ID NO: 83 - protein, 20 aa, Homo sapiens
  Domain: CDR3 amino acid sequence of BV16 clonotype derived from ST specimen of RA patient
YFCASSQLAGPYNEQFFGPG

SEQ ID NO: 84 - DNA, 60 bp, Artificial Sequence
  CDS: CDR3 nucleic acid sequence of BV16 clonotype derived from ST specimen of RA patients
tatttctgtgccagcagccagctagcgggaccctacaatgagcagttcttcgggccagga

SEQ ID NO: 85 - protein, 20 aa, Homo sapiens
  Domain: CDR3 amino acid sequence of BV16 clonotype derived from ST specimen of RA patient
YFCASSLLGTVSYEQFFGPG

SEQ ID NO: 86 - DNA, 60 bp, Artificial Sequence
  CDS: CDR3 nucleic acid sequence of BV16 clonotype derived from ST specimen of RA patients
tatttctgtgccagcagccttctcggcacagtatcctatgagcagttcttcgggccaggc

SEQ ID NO: 87 - protein, 20 aa, Homo sapiens
  Domain: CDR3 amino acid sequence of BV16 clonotype derived from ST specimen of RA patient
YFCASPLGTALSYEQFFGPG

SEQ ID NO: 88 - DNA, 60 bp, Artificial Sequence
  CDS: CDR3 nucleic acid sequence of BV16 clonotype derived from ST specimen of RA patients
tatttctgtgccagcccccttgggacagcgctatcctacgagcagtacttcgggccgggc

SEQ ID NO: 89 - protein, 20 aa, Homo sapiens
  Domain: CDR3 amino acid sequence of BV16 clonotype derived from ST specimen of RA patient
YFCASSQADGTHYEQFFGPG

SEQ ID NO: 90 - DNA, 60 bp, Artificial Sequence
  CDS: CDR3 nucleic acid sequence of BV16 clonotype derived from ST specimen of RA patients
tatttctgtgccagcagccaagctgacgggacccattacgagcagtacttcgggccgggc

SEQ ID NO: 91 - protein, 20 aa, Homo sapiens
  Domain: CDR3 amino acid sequence of BV16 clonotype derived from ST specimen of RA patient
YFCASSQDKGHFYEQFFGPG

SEQ ID NO: 92 - DNA, 60 bp, Artificial Sequence
  CDS: CDR3 nucleic acid sequence of BV16 clonotype derived from ST specimen of RA patients
tatttctgtgccagcagccaagataagggacacttctacgagcagtacttcgggccgggc

SEQ ID NO: 93 - protein, 20 aa, Homo sapiens
  Domain: CDR3 amino acid sequence of BV16 clonotype derived from ST specimen of RA patient
YFCASSQADGTHYEQFFGPG

SEQ ID NO: 94 - DNA, 60 bp, Artificial Sequence; identical to SEQ ID NO: 90
  CDS: CDR3 nucleic acid sequence of BV16 clonotype derived from ST specimen of RA patients

SEQ ID NO: 95 - protein, 20 aa, Homo sapiens
  Domain: CDR3 amino acid sequence of BV16 clonotype derived from ST specimen of RA patient
YFCASSWGGTDIYEQFFGPG

SEQ ID NO: 96 - DNA, 60 bp, Artificial Sequence
  CDS: CDR3 nucleic acid sequence of BV16 clonotype derived from ST specimen of RA patients
tatttctgtgccagcagctggggcgggacagacatctacgagcagtacttcgggccgggc

SEQ ID NO: 97 - protein, 20 aa, Homo sapiens
  Domain: CDR3 amino acid sequence of BV16 clonotype derived from ST specimen of RA patient
YFCASSLLGTVSYEQFFGPG

SEQ ID NO: 98 - DNA, 60 bp, Artificial Sequence
  CDS: CDR3 nucleic acid sequence of BV16 clonotype derived from ST specimen of RA patients
tatttctgtgccagcagccttctcggcacagtatcctacgagcagtacttcgggccgggc

SEQ ID NO: 99 - protein, 18 aa, Homo sapiens
  Domain: CDR3 amino acid sequence of BV16 clonotype derived from ST specimen of RA patient
YFCASSQGLNTEAFFGQG

SEQ ID NO: 100 - DNA, 54 bp, Artificial Sequence
  CDS: CDR3 nucleic acid sequence of BV16 clonotype derived from ST specimen of RA patients
tatttctgtgccagcagccaaggccttaacactgaagctttctttggacaaggc

SEQ ID NO: 101 - protein, 18 aa, Homo sapiens
  Domain: CDR3 amino acid sequence of BV16 clonotype derived from ST specimen of RA patient
YFCASRASRYTEAFFGQG

SEQ ID NO: 102 - DNA, 54 bp, Artificial Sequence
  CDS: CDR3 nucleic acid sequence of BV16 clonotype derived from ST specimen of RA patients
tatttctgtgccagcagggcaagcaggtacactgaagctttctttggacaaggc

SEQ ID NO: 103 - protein, 18 aa, Homo sapiens
  Domain: CDR3 amino acid sequence of BV16 clonotype derived from ST specimen of RA patient
YFCASRASRYTEAFFGQG

SEQ ID NO: 104 - DNA, 54 bp, Artificial Sequence; identical to SEQ ID NO: 102
  CDS: CDR3 nucleic acid sequence of BV16 clonotype derived from ST specimen of RA patients

SEQ ID NO: 105 - protein, 18 aa, Homo sapiens
  Domain: CDR3 amino acid sequence of BV16 clonotype derived from ST specimen of RA patient
YFCASSTGVNTEAFFGQG

SEQ ID NO: 106 - DNA, 54 bp, Artificial Sequence
  CDS: CDR3 nucleic acid sequence of BV16 clonotype derived from ST specimen of RA patients
tatttctgtgccagcagtacaggggtgaacactgaagctttctttggacaaggc

SEQ ID NO: 107 - protein, 18 aa, Homo sapiens
  Domain: CDR3 amino acid sequence of BV16 clonotype derived from ST specimen of RA patient
YFCASSLTTNTEAFFGQG

SEQ ID NO: 108 - DNA, 54 bp, Artificial Sequence
  CDS: CDR3 nucleic acid sequence of BV16 clonotype derived from ST specimen of RA patients
tatttctgtgccagcagcctcacaacgaacactgaagctttctttggacaaggc

SEQ ID NO: 109 - protein, 18 aa, Homo sapiens
  Domain: CDR3 amino acid sequence of BV16 clonotype derived from ST specimen of RA patient
YFCASSQDSYTEAFFGQG

SEQ ID NO: 110 - DNA, 54 bp, Artificial Sequence
  CDS: CDR3 nucleic acid sequence of BV16 clonotype derived from ST specimen of RA patients
tatttctgtgccagcagccaagattcgtacactgaagctttctttggacaaggc

SEQ ID NO: 111 - protein, 18 aa, Homo sapiens
  Domain: CDR3 amino acid sequence of BV16 clonotype derived from ST specimen of RA patient
YFCASSWGGNTEAFFGQG

SEQ ID NO: 112 - DNA, 54 bp, Artificial Sequence
  CDS: CDR3 nucleic acid sequence of BV16 clonotype derived from ST specimen of RA patients
tatttctgtgccagcagctgggggggcaacactgaagctttctttggacaaggc

SEQ ID NO: 113 - protein, 20 aa, Homo sapiens
  Domain: CDR3 amino acid sequence of BV14 clonotype derived from ST specimen of RA patients
YFCASSPTRDRGNEQFFGPG

SEQ ID NO: 114 - DNA, 63 bp, Artificial Sequence
  CDS: CDR3 nucleic acid sequence of BV14 clonotype derived from ST specimen of RA patients
tacttctgtgccagcagtcccacgcgggacaggggaaataatgagcagttcttcgggccagga

SEQ ID NO: 115 - protein, 22 aa, Homo sapiens
  Domain: CDR3 amino acid sequence of BV14 clonotype derived from ST specimen of RA patients
YFCASSSPIAGSSYNEQFFGPG

SEQ ID NO: 116 - DNA, 63 bp, Artificial Sequence
  CDS: CDR3 nucleic acid sequence of BV14 clonotype derived from ST specimen of RA patients
tacttctgtgccagcagttccccaatagcggggagctccaatgagcagttcttcgggccagga

SEQ ID NO: 117 - protein, 20 aa, Homo sapiens
  Domain: CDR3 amino acid sequence of BV14 clonotype derived from ST specimen of RA patients
YFCASSFWAPTDNEQFFGPG

SEQ ID NO: 118 - DNA, 63 bp, Artificial Sequence
  CDS: CDR3 nucleic acid sequence of BV14 clonotype derived from ST specimen of RA patients
tacttctgtgccagcagtttctgggcccctacggacaataatgagcagttcttcgggccagga

SEQ ID NO: 119 - protein, 21 aa, Homo sapiens
  Domain: CDR3 amino acid sequence of BV14 clonotype derived from ST specimen of RA patients
YFCASSSSSPTSYNEQFFGPG

SEQ ID NO: 120 - DNA, 60 bp, Artificial Sequence
  CDS: CDR3 nucleic acid sequence of BV14 clonotype derived from ST specimen of RA patients
tacttctgtgccagcagttctagcagccccacctcctacgagcagttcttcgggccagga

SEQ ID NO: 121 - protein, 20 aa, Homo sapiens
  Domain: CDR3 amino acid sequence of BV14 clonotype derived from ST specimen of RA patients
YFCASSPREGLLNEQFFGPG

SEQ ID NO: 122 - DNA, 63 bp, Artificial Sequence
  CDS: CDR3 nucleic acid sequence of BV14 clonotype derived from ST specimen of RA patients
tacttctgtgccagcagccctagggagggcctcctcaataatgagcagttcttcgggccagga

SEQ ID NO: 123 - protein, 21 aa, Homo sapiens
  Domain: CDR3 amino acid sequence of BV14 clonotype derived from ST specimen of RA patients
YFCASSPWTSGSGNEQFFGPG

SEQ ID NO: 124 - DNA, 60 bp, Artificial Sequence
  CDS: CDR3 nucleic acid sequence of BV14 clonotype derived from ST specimen of RA patients
tacttctgtgccagcagtccctggactagcgggagtggtgagcagttcttcgggccagga

SEQ ID NO: 125 - protein, 19 aa, Homo sapiens
  Domain: CDR3 amino acid sequence of BV14 clonotype derived from ST specimen of RA patients
YFCASSLRTRFYEQYFGPG

SEQ ID NO: 126 - DNA, 57 bp, Artificial Sequence
  CDS: CDR3 nucleic acid sequence of BV14 clonotype derived from ST specimen of RA patients
tacttctgtgccagcagtttaaggacacgcttctacgagcagttcttcgggccagga

SEQ ID NO: 127 - protein, 20 aa, Homo sapiens
  Domain: CDR3 amino acid sequence of BV14 clonotype derived from ST specimen of RA patients
YFCASSLTSGRQYEQYFGPG

SEQ ID NO: 128 - DNA, 60 bp, Artificial Sequence
  CDS: CDR3 nucleic acid sequence of BV14 clonotype derived from ST specimen of RA patients
tacttctgtgccagcagtttgaccagcgggcgtcagtacgagcagttcttcgggccagga

SEQ ID NO: 129 - protein, 20 aa, Homo sapiens
  Domain: CDR3 amino acid sequence of BV14 clonotype derived from ST specimen of RA patients
YFCASSSGGSLFYEQYFGPG

SEQ ID NO: 130 - DNA, 60 bp, Artificial Sequence
  CDS: CDR3 nucleic acid sequence of BV14 clonotype derived from ST specimen of RA patients
tacttctgtgccagcagttccgggggcagtctgttctacgagcagttcttcgggccagga

SEQ ID NO: 131 - protein, 20 aa, Homo sapiens
  Domain: CDR3 amino acid sequence of BV14 clonotype derived from ST specimen of RA patients
YFCASSLSVGATYEQYFGPG

SEQ ID NO: 132 - DNA, 60 bp, Artificial Sequence
  CDS: CDR3 nucleic acid sequence of BV14 clonotype derived from ST specimen of RA patients
tacttctgtgccagcagtttatcggtcggggctacctacgagcagttcttcgggccagga

SEQ ID NO: 133 - protein, 20 aa, Homo sapiens
  Domain: CDR3 amino acid sequence of BV14 clonotype derived from ST specimen of RA patients
YFCASSSGGSLFYEQYFGPG

SEQ ID NO: 134 - DNA, 60 bp, Artificial Sequence; identical to SEQ ID NO: 130
  CDS: CDR3 nucleic acid sequence of BV14 clonotype derived from ST specimen of RA patients

SEQ ID NO: 135 - protein, 20 aa, Homo sapiens
  Domain: CDR3 amino acid sequence of BV14 clonotype derived from ST specimen of RA patients
YFCASSPSISSHYEQYFGPG

SEQ ID NO: 136 - DNA, 60 bp, Artificial Sequence
  CDS: CDR3 nucleic acid sequence of BV14 clonotype derived from ST specimen of RA patients
tacttctgtgccagcagcccaagtattagttcccactacgagcagttcttcgggccagga

SEQ ID NO: 137 - protein, 19 aa, Homo sapiens
  Domain: CDR3 amino acid sequence of BV14 clonotype derived from ST specimen of RA patients
YFCASSRDGVSYEQYFGPG

SEQ ID NO: 138 - DNA, 57 bp, Artificial Sequence
  CDS: CDR3 nucleic acid sequence of BV14 clonotype derived from ST specimen of RA patients
tacttctgtgccagcagtcgtgatggggtctcctacgagcagttcttcgggccagga

SEQ ID NO: 139 - protein, 19 aa, Homo sapiens
  Domain: CDR3 amino acid sequence of BV14 clonotype derived from ST specimen of RA patients
YFCASSLSSTGREQYFGPG

SEQ ID NO: 140 - DNA, 57 bp, Artificial Sequence
  CDS: CDR3 nucleic acid sequence of BV14 clonotype derived from ST specimen of RA patients
tacttctgtgccagcagtttatcttcgacagggagggagcagtacttcgggccgggc

SEQ ID NO: 141 - protein, 20 aa, Homo sapiens
  Domain: CDR3 amino acid sequence of BV14 clonotype derived from ST specimen of RA patients
YFCASSLSFRLDYEQYFGPG

SEQ ID NO: 142 - DNA, 60 bp, Artificial Sequence
  CDS: CDR3 nucleic acid sequence of BV14 clonotype derived from ST specimen of RA patients
tacttctgtgccagcagtttatcgtttagactagactacgagcagttcttcgggccagga

SEQ ID NO: 143 - protein, 20 aa, Homo sapiens
  Domain: CDR3 amino acid sequence of BV14 clonotype derived from ST specimen of RA patients
YFCASSPSGQGSYEQYFGPG

SEQ ID NO: 144 - DNA, 60 bp, Artificial Sequence
  CDS: CDR3 nucleic acid sequence of BV14 clonotype derived from ST specimen of RA patients
tacttctgtgccagcagtccgtcgggacaggggtcctacgagcagttcttcgggccagga

SEQ ID NO: 145 - protein, 20 aa, Homo sapiens
  Domain: CDR3 amino acid sequence of BV14 clonotype derived from ST specimen of RA patients
YFCASSFGTVLSYEQYFGPG

SEQ ID NO: 146 - DNA, 60 bp, Artificial Sequence
  CDS: CDR3 nucleic acid sequence of BV14 clonotype derived from ST specimen of RA patients
tacttctgtgccagcagttttgggacagtcctctcctacgagcagttcttcgggccagga

SEQ ID NO: 147 - protein, 20 aa, Homo sapiens
  Domain: CDR3 amino acid sequence of BV14 clonotype derived from ST specimen of RA patients
YFCASSPRLAGDKEQYFGPG

SEQ ID NO: 148 - DNA, 61 bp, Artificial Sequence
  CDS: CDR3 nucleic acid sequence of BV14 clonotype derived from ST specimen of RA patients
tacttctgtgccagcagtccccgactagcgggagataaaggagcagtacttcgggccgggc

SEQ ID NO: 149 - protein, 20 aa, Homo sapiens
  Domain: CDR3 amino acid sequence of BV14 clonotype derived from ST specimen of RA patients
YFCASSLSARTTYEQYFGPG

SEQ ID NO: 150 - DNA, 60 bp, Artificial Sequence
  CDS: CDR3 nucleic acid sequence of BV14 clonotype derived from ST specimen of RA patients
tacttctgtgccagcagtttaagtgccaggacaacctacgagcagttcttcgggccagga

SEQ ID NO: 151 - protein, 19 aa, Homo sapiens
  Domain: CDR3 amino acid sequence of BV14 clonotype derived from ST specimen of RA patients
YFCASSLIGGNEKLFLGSG

SEQ ID NO: 152 - DNA, 57 bp, Artificial Sequence
  CDS: CDR3 nucleic acid sequence of BV14 clonotype derived from ST specimen of RA patients
tacttctgtgccagcagtttgatagggggcaatgaaaaactgttttttggcagtgga

SEQ ID NO: 153 - protein, 18 aa, Homo sapiens
  Domain: CDR3 amino acid sequence of BV14 clonotype derived from ST specimen of RA patients
YFCASSLSQETEAFFGQG

SEQ ID NO: 154 - DNA, 53 bp, Artificial Sequence
  CDS: CDR3 nucleic acid sequence of BV14 clonotype derived from ST specimen of RA patients
tacttctgtgccagagtttatcccaggaaactgaagctttctttggacaaggc

SEQ ID NO: 155 - protein, 19 aa, Homo sapiens
  Domain: CDR3 amino acid sequence of BV14 clonotype derived from ST specimen of RA patients
YFCASRAGTGFEKLFFGSG

SEQ ID NO: 156 - DNA, 54 bp, Artificial Sequence
  CDS: CDR3 nucleic acid sequence of BV14 clonotype derived from ST specimen of RA patients
tacttctgtgccagcagggccgggacagggtttaaactgttttttggcagtgga

SEQ ID NO: 157 - protein, 18 aa, Homo sapiens
  Domain: CDR3 amino acid sequence of BV14 clonotype derived from ST specimen of RA patients
YFCASSLSQNTEAFFGQG

SEQ ID NO: 158 - DNA, 54 bp, Artificial Sequence
  CDS: CDR3 nucleic acid sequence of BV14 clonotype derived from ST specimen of RA patients
tacttctgtgccagcagtctgtcacagaacactgaagctttctttggacaaggc

SEQ ID NO: 159 - protein, 18 aa, Homo sapiens
  Domain: CDR3 amino acid sequence of BV14 clonotype derived from ST specimen of RA patients
YFCASSPRVNTEAFFGQG

SEQ ID NO: 160 - DNA, 53 bp, Artificial Sequence
  CDS: CDR3 nucleic acid sequence of BV14 clonotype derived from ST specimen of RA patients
tacttctgtgccagagtccccgggtcaacactgaagctttctttggacaaggc

SEQ ID NO: 161 - protein, 18 aa, Homo sapiens
  Domain: CDR3 amino acid sequence of BV14 clonotype derived from ST specimen of RA patients
YFCASSLSQETEAFFGQG

SEQ ID NO: 162 - DNA, 53 bp, Artificial Sequence; identical to SEQ ID NO: 154
  CDS: CDR3 nucleic acid sequence of BV14 clonotype derived from ST specimen of RA patients

SEQ ID NO: 163 - protein, 18 aa, Homo sapiens
  Domain: CDR3 amino acid sequence of BV14 clonotype derived from ST specimen of RA patients
YFCASSLGRNTEAFFGQG

SEQ ID NO: 164 - DNA, 54 bp, Artificial Sequence
  CDS: CDR3 nucleic acid sequence of BV14 clonotype derived from ST specimen of RA patients
tacttctgtgccagcagcctagggaggaacactgaagctttctttggacaaggc

SEQ ID NO: 165 - protein, 18 aa, Homo sapiens
  Domain: CDR3 amino acid sequence of BV14 clonotype derived from ST specimen of RA patients
YFCASSSRGYTEAFFGQG

SEQ ID NO: 166 - DNA, 54 bp, Artificial Sequence
  CDS: CDR3 nucleic acid sequence of BV14 clonotype derived from ST specimen of RA patients
tacttctgtgccagcagttccaggggatacactgaagctttctttggacaaggc

SEQ ID NO: 167 - protein, 18 aa, Homo sapiens
  Domain: CDR3 amino acid sequence of BV14 clonotype derived from ST specimen of RA patients
YFCASSSLATAEAFFGQG

SEQ ID NO: 168 - DNA, 54 bp, Artificial Sequence
  CDS: CDR3 nucleic acid sequence of BV14 clonotype derived from ST specimen of RA patients
tacttctgtgccagcagttccctcgctactgctgaagctttctttggacaaggc

(19) United States
(12) Patent Application Publication
(10) Pub. No.: US 2005/0010030 A1
(43) Pub. Date: Jan. 13, 2005

(54) T cell receptor CDR3 sequence and methods for detecting and treating rheumatoid arthritis

(76) Inventors: Jingwu Z. Zang, Missouri City, TX (US); Walter Kowk Keung Ho, Taipo (CN); Dongqing Zhang, Shanghai (CN); Wei Sun, Houston, TX (US)

Correspondence Address: Attention: J. Wendy Davis, Ph.D.;BRACEWELL & PATTERSON, L.L.P., P.O. Box 61389, Houston, TX, 77208-1389, US

(21) Appl. No.: 10/612,468

(22) Filed: Jul. 2, 2003

(51) Int. Cl. C07K 14/705; C07H 21/04; A61K 39/00

(52) U.S. Cl. 530/350; 536/23.5; 435/69.1; 435/320.1; 435/325; 424/185.1

(57) ABSTRACT

A substantially pure and isolated DNA fragment having a nucleic acid sequence as shown in SEQ ID NO. 1 or SEQ ID NO. 2, and a substantially pure peptide having an amino acid sequence selected from the group consisting of SEQ ID NO. 3, SLS, SEQ ID NO. 4, SQD, SLL and SEQ ID NO. 5 are provided. Also provided are vaccines, antibodies and pharmaceutical compositions generated from at least one of the DNA fragments and/or peptides. Further provided are methods for detecting and/or treating rheumatoid arthritis.

### BACKGROUND OF THE INVENTION

1. Field of the Invention

The present invention generally relates to the field of molecular biology and medicine. More particularly, the present invention relates to T cell receptor specific CDR3 sequence and methods for diagnosing and treating rheumatoid arthritis.

2. Description of Related Art

The receptors recognizing antigens at the surface of mature T lymphocytes (T-cell antigen receptors or TCRs) possess a structure having a certain similarity with those of immunoglobulins. Therefore, they contain heterodimeric structures comprising α and β glycoprotein chains or γ and δ glycoprotein chains.

The directory of T-cell receptors must be able to address the immense diversity of antigenic determinants. This is obtained by genetic recombination of different discontinuous segments of genes that code for the different structural regions of T-cell receptors. Thus, the genes contain V segments (variable segments), optionally D segments (diversity segments), J segments (junction segments) and C segments (constant segments). During the differentiation of T-cells, specific genes are created by recombination of V, D and J segments for the β and δ loci and V and J segments for the α and β loci. These specific combinations as well as the pairing of two chains create the combinational diversity. This diversity is highly amplified by two supplementary mechanisms, namely the imprecise recombination of V-D-J or V-J segments and the addition of nucleotides corresponding to the N region (Davis et al., 1988). The genes encoding the T-cell receptor (TCR) α and β chains are produced by the combination of the Vα, Jα and Cα and Vβ, Jβ, Dβ, and Cβ segments respectively.

More than 70 Vα and Vβ gene segments have been molecularly characterized and are classified into 29 and 25 subfamilies, respectively, on the basis of sequence similarity in their coding regions. These distinct levels of TCR diversity allow the generation of a large T cell repertoire which is able to face the large diversity of short peptide bound to the MHC molecules. Hypervariable complementary determining region-3 (CDR3)-like loops encoded by V(D)J junctions are thought to interact directly with the antigenic peptide. The characterization of TCR polypeptides is a way to precisely analyze T cell responses.

Rheumatoid arthritis (RA) is characterized by chronic inflammation of the synovium of the peripheral joints, in which T cells are thought to play an important role in the pathogenesis (Lee et al., 2001 and Lipsky et al., 1998). This is supported by marked infiltration and accumulation of Th1 pro-inflammatory cells in the synovial membrane of RA in close association with MHC Class II genes, including DR4 (genotypes B1*0404 and DRB1*0401) and DQ (DQB1*0302 and DQB1*0301), in Caucasian RA patients (Kerlan-Candon et al., 2001; MacGregor et al., 1995; and Fries et al., 2002). Further supporting evidence includes skewing of cytokine environment in favor of T cell-mediated inflammation and clonal expansion of infiltrating T cells in the affected joints (Dolhain et al., 1996; Berner et al., 2000; Davis et al., 2001; Goronzy et al., 1994; Struyk et al., 1993; Gonzalez-Quintial et al., 1996; and Alam et al., 1996). However, the antigen specificity of the infiltrating T cells in rheumatoid synovium is unknown. Several self antigens, including collagen type II, heat shock proteins and others, are implicated in RA based on T cell reactivity to these antigens in patients with RA (Londei et al., 1989; Pope et al., 1989; Devereux et al., 1991; and Res et al., 1994). Microbial antigens, such as mycobacterial antigens and staphylococcal superantigens, may also contribute to T cell activation in RA (Paliard et al., 1991 and Holoshitz et al., 1986). However, it is unclear whether T cell responses to any of these antigens are clinically relevant to RA.

In the absence of an eliciting antigen(s) associated with RA, attempts have been made to identify T cell receptor (TCR) structural features characteristic of infiltrating T cells derived from synovial fluid or synovial membrane of RA patients. It was hoped that a common TCR structural feature(s) or characteristic clonotypes of T cells associated with rheumatoid synovium may provide better understanding of the mechanism whereby these infiltrating T cells are activated and perpetuated in the synovium and may potentially lead to novel therapeutic strategies. As the T cell receptor repertoire is shaped by the genetic background of the individual and the response to self or environmental antigens, antigen-driven stimulation in the context of similar MHC Class II molecules leads to oligoclonal expansion of T cells utilizing common V-D-J segments. On the other hand, T cell activation induced by superantigen stimulation is characterized by polyclonal expansion of a particular TCR BV gene family with different D-J segments. Therefore, it is important to delineate the BV gene distribution pattern and structural features of the third complementarity-determining region (CDR3) among T cells in the rheumatoid synovium.

A number of studies in Caucasian RA patients have shown that TCR BV usage of T cells derived from synovial fluid and, in some cases, synovial membranes of RA patients is variably skewed to certain BV genes, including BV14, BV17 and several others (Zagon et al., 1994; Alam et al., 1995; VanderBorght et al., 2000; Jenkins et al., 1993; and Williams et al., 1992). Analysis of CDR3 of over-expressed BV genes has revealed some clonotypes that only exist in rheumatoid synovium but not peripheral T cells, suggesting T cell clonal expansion in the affected joints in RA (Li et al., 1994; Mima et al., 1999; and Davey et al., 1997). However, clonality and TCR BV gene usage of infiltrating T cells in RA synovial fluid or membranes are relatively heterogeneous, which complicates BV gene analysis using regular or semi-quantitative PCR. This may be attributable to significant variations in the detection of over-expressed BV genes and CDR3 structural features of RA synovial T cells reported in different studies. Furthermore, relatively diverse clonality of infiltrating T cells seen in RA has significantly increased difficulties in the identification of common CDR3 structural features. Multiple CDR3 sizing peaks typically found in synovial TCR transcripts of RA require several hundreds to thousands of measurements for each sample when multiplied by the number of BV and BJ genes (25 BV and 13 BJ genes), to characterize clonotypes of interest (Even et al., 1995).

U.S. Pat. No. 6,159,470 discloses a method of treating rheumatoid arthritis in a human individual comprising binding Vβ17 containing T cells in the individual with an effective amount of a cytotoxic or cytostatic agent specifically reactive with Vβ17 to kill or inhibit proliferation of the T cells, wherein the agent is an antibody.

U.S. Pat. No. 5,985,552 discloses a method of diagnosing or predicting susceptibility to rheumatoid arthritis in an individual comprising selectively detecting levels in a sample isolated from the individual of T cells having on their surface Vβ14- or Vβ17-containing T cell receptors, the presence of abnormal levels of said Vβ14- or Vβ17-containing T cell receptors compared to levels in normal individuals indicating rheumatoid arthritis or susceptibility to rheumatoid arthritis.

U.S. Pat. No. 6,207,645 discloses a method for eliciting an immune response in an individual suffering from rheumatoid arthritis, comprising administering directly into muscle tissue of the individual a plasmid vector comprising a promoter operably linked to a nucleic acid sequence encoding a single chain T cell receptor variable beta 3, 14 or 17 peptide, or fragments thereof, wherein the nucleic acid sequence is expressed in the muscle tissue at a level sufficient to elicit an immune response against the encoded peptide in the individual.

U.S. Pat. No. 6,221,352 discloses a method of preventing the proliferation of Vβ14-expressing T cells in a human individual having rheumatoid arthritis, comprising administering to the individual an effective amount of a cytotoxic or cytostatic agent, wherein the agent comprises an antibody, and wherein the antibody selectively binds Vβ14 expressed by the T cells.

Mima, et al. reports that a dominant CDR3 sequence, CASS-PRERAT-YEQ, was found in Vbeta14⁺ T cells from the rheumatoid joint of two different patients. Common TCR structural feature(s) or characteristic clonotypes of T cells associated with rheumatoid synovium may provide better understanding of the mechanism whereby these infiltrating T cells are activated and perpetuated in the synovium and may potentially lead to novel therapeutic strategies. Therefore, there is a long-standing need for identifying TCR sequences characteristics of T cells associated with rheumatoid arthritis patients and for an effective method to diagnose and treat rheumatoid arthritis. The present invention fulfills this long-standing need in the art.

### SUMMARY OF INVENTION

The present invention is directed to a substantially pure and isolated DNA fragment comprising a nucleic acid sequence as shown in SEQ ID NO. 1 or SEQ ID NO. 2, which is part of the complementary determining region-3 (CDR3) in the Vβ14 family (BV14 gene) and Vβ16 family (BV16 gene) of T cell receptors in patients with rheumatoid arthritis (RA), respectively.

The present invention is also directed to a vaccine which comprises at least one DNA fragment selected from the group consisting of SEQ ID NO. 1 and SEQ ID NO. 2.

The present invention is also directed to a substantially pure and isolated peptide having an amino acid sequence selected from the group consisting of SEQ ID NO. 3, SLS, SEQ ID NO. 4, SQD, SLL and SEQ ID NO. 5, which are derived from the CDR3 of T cell receptor beta-chain BV14 (SEQ ID NO. 3 and SLS) or BV16 (SEQ ID NO. 4, SQD, SLL and SEQ ID NO. 5) gene in an individual suffering from rheumatoid arthritis. Also provided is an antibody directed against such peptide.

The present invention is also directed to a vaccine which comprises at least one peptide having an amino acid sequence derived from the CDR3 of a T cell receptor gene selected from the group consisting of BV14 and BV16 in an individual suffering from rheumatoid arthritis.

The present invention is further directed to a method for detecting rheumatoid arthritis. This method advantageously includes obtaining a tissue sample from the suspected individual and a normal individual, respectively; measuring the expression level of BV14 and/or BV16 of T cell receptors in the tissue sample; and comparing the expression level in the suspected and normal individuals. If BV14 and/or BV16 are expressed in a substantially higher level in the suspected individual than in the normal individual, it is indicated that the individual might have rheumatoid arthritis.

The present invention is further directed to a method for detecting rheumatoid arthritis in an individual of Chinese population. This method advantageously includes obtaining a tissue sample from the suspected individual and a normal individual, respectively; measuring the expression level of BV16 of T cell receptors in the tissue sample; and comparing the expression level in the suspected and normal individuals. If BV16 is expressed in a substantially higher level in the suspected individual than in the normal individual, it is indicated that the individual of Chinese population might have rheumatoid arthritis.

The present invention is further directed to a method for detecting rheumatoid arthritis. This method advantageously includes generating a probe complementary to a DNA fragment having a nucleic acid sequence selected from the group consisting of SEQ ID NO. 1 and SEQ ID NO. 2; obtaining a tissue sample from the suspected individual; and mixing the probe with the tissue sample. In this method a positive hybridization signal indicates a possible detection of rheumatoid arthritis in the suspected individual.

The present invention is still further directed to a method for detecting rheumatoid arthritis. This method advantageously includes generating an antibody directed against a peptide having an amino acid sequence selected from the group consisting of SEQ ID NO. 3, SLS, SEQ ID NO. 4, SQD, SLL and SEQ ID NO. 5; obtaining a tissue sample from the suspected individual; and mixing the antibody with the tissue sample. In this method a positive signal indicates a possible detection of rheumatoid arthritis in the suspected individual.

The present invention is yet further directed to a method for treating rheumatoid arthritis by administering to the individual with an effective amount of an immunogenic T cell receptor peptide to elicit an immune response. Such peptide comprises an amino acid sequence selected from the group consisting of SEQ ID NO. 3, SLS, SEQ ID NO. 4, SQD, SLL and SEQ ID NO. 5.

The present invention is yet further directed to a method for treating rheumatoid arthritis by administering to the individual with an effective amount of an antibody directed against a peptide having an amino acid sequence selected from the group consisting of SEQ ID NO. 3, SLS, SEQ ID NO. 4, SQD, SLL and SEQ ID NO. 5.

The present invention is still yet further directed to a method for treating rheumatoid arthritis. This method advantageously includes administering to the individual with a DNA expression vector comprising a promoter operably linked to a DNA fragment having a nucleic acid sequence encoding a single chain T cell receptor variable beta 16 (Vβ16) peptide, or fragments thereof, and then expressing the DNA fragment in the individual In this method, the DNA fragment is expressed at a level sufficient to elicit an immune response against the encoded peptide thereby preventing onset of rheumatoid arthritis or treating rheumatoid arthritis in the individual.

The present invention is still yet further directed to a method for treating rheumatoid arthritis. This method advantageously includes administering to the individual with a DNA expression vector comprising a promoter operably linked to a DNA fragment having a nucleic acid sequence encoding a single chain T cell receptor variable beta 14 (Vβ14) peptide, or fragments thereof, and then expressing the DNA fragment in the individual In this method, the nucleic acid sequence comprises a sequence as shown in SEQ ID NO. 1. Upon entering the individual, the DNA fragment is expressed at a level sufficient to elicit an immune response against the encoded peptide thereby preventing onset of rheumatoid arthritis or treating rheumatoid arthritis in the individual.

The present invention is still yet further directed to a pharmaceutical composition for suppressing pathogenic T cell response in an individual suffering from rheumatoid arthritis. This composition advantageously comprises an immunologically effective amount of a peptide derived from a single chain T cell receptor variable beta 14 (Vβ14) or 16 (Vβ16), or fragments thereof, and a pharmaceutically acceptable carrier.

The foregoing and other advantages of the present invention will be apparent to those skilled in the art, in view of the following detailed description of the preferred embodiment of the present invention, taken in conjunction with the accompanying drawings.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1A shows the PCR amplification efficiency with a set of oligonucleotide primers specific for 25 BV family and BC gene. FIG. 1B shows the results for real-time PCR analysis of peripheral blood mononuclear cells prepared separately from four healthy individuals and cultured in the presence and absence of toxic shock syndrome toxin.

FIG. 2 shows a highly significant BV skewing for BV14 (mean expression level of 27%), BV16 (mean expression level of 31%) and, to a lesser extent, BV20 (17%) in RA-derived synovial lesion tissues (ST) specimens.

FIG. 3 shows that BV14 gene exhibits heterogeneous CDR3 length profile in both synovial fluid (SF) and ST specimens derived from RA patients when a pair of 5′BV14-3′BC specific primers were used to analyze the sequence regions between BV14-BJ-3′BC.

FIG. 4 show that BV16 genes exhibited heterogeneous CDR3 length profile in both SF and ST specimens derived from RA patients when a pair of 5′BV16-3′BC specific primers were used to analyze the sequence regions between BV16-BJ-3′BC.

FIG. 5 shows the results of BV14 and BV16 transcripts analyzed for CDR3 length profile by immunoscope using BV14 or BV16 primers and a set of primers specific for 13 individual BJ genes, respectively.

FIG. 6 shows representative clonotype patterns, which have the same BV and BJ combinations with similar CDR3 length.

### DETAILED DESCRIPTION OF THE INVENTION

To aid in understanding the invention, the following terms have the definitions as set forth below.

“PCR” means the polymerase chain reaction, for example, as generally described in U.S. Pat. No. 4,683,202. PCR is an amplification technique wherein selected oligonucleotides, or primers, are hybridized to nucleic acid templates in the presence of a polymerization agent (such as polymerase) and four nucleotide triphosphates, and extension products are formed from the primers. These products are then denatured and used as templates in a cycling reaction that amplifies the number and amount of existing nucleic acids to facilitate their subsequent detection. A variety of PCR techniques are available and may be used with the methods according to the invention.

“Primer” means an oligonucleotide, whether natural or synthetic, capable of acting as a point of initiation of DNA synthesis complementary to a specific DNA sequence on a template molecule.

“Superantigens” means antigens or fragments thereof that bind preferentially to T cells at specific sites on the β chain of a T cell receptor (TCR) and stimulate T cells at very high frequency rate. Superantigens activate T cells by binding to specific Vβs. The superantigen binding sites of various TCRs have been distinguished from the conventional hypervariable complementary determining regions (CDRs) of TCRs. These CDRs represent the regions of TCRs thought to be responsible for binding conventional antigens that are complexed to MHC.

“Vβ14” refers to a specific human β chain variable region of T cell receptors. Vβ14 has the following amino acid sequence:

| | |
|---|---|
| MGPQLLGYVVLCLLGAGPLEAQVTQNPRYLITVTGKKLTVTCSQ | (SEQ ID NO. 6) |
| NMNHEYMSWYRQDPGLGLRQIYYSMNVEVTDKGDVPEGYKVSRK | |
| EKRNFPLILESPSPNQTSLYFCASS. | |

“Vβ16” refers to a specific human β chain variable region of T cell receptors. Vβ16 has the following amino acid sequence:

| | |
|---|---|
| IEAGVTQFPSHSVIEKGQTV TLRCDPISGHDNLYWYRRVMGKE | (SEQ ID NO. 7) |
| IKFLLHFVKESKQDESGMPNNRFLAERTGGTYSTLKVQPAELED | |
| SGVYFCASS. | |

“Fragment” means an immunogenically effective subset of the amino acid sequence that comprises a T cell receptor (TCR). The term is intended to include such fragments in conjunction with or combined with additional sequences or moieties, for example, where the peptide is coupled to other amino acid sequences or to a carrier.

“Complementary determining region-3 (CDR3)” is also known as the V(D)J region. Due to the recombination of the V, D and J region genes prior to maturation, the amino acid sequence across these regions is virtually unique to each T cell and its clones. CDR3 or fragment thereof is useful as a vaccine of the present invention since T cell immunity elicited by peptides corresponding to this region is expected to be highly specific for a particular antigen.

The present invention examined the BV usage pattern of infiltrating T cells derived from synovial material of a cohort of Chinese RA patients, whose human leukocyte antigen (HLA) background differed from that of Caucasian patients, to determine the potential association of BV gene distribution with HLA. Common clonotypes and CDR3 structural characteristics were identified in over-expressed BV transcripts of synovial T cells from different RA patients. The analyses have been performed with TCR transcripts derived ex vivo from synovial material and blood of patients. In vitro culture was not required, which would be likely to introduce biases. It is believed that infiltrating T cells in the rheumatoid synovium are driven and shaped by common self or microbial antigens in the context of RA-associated DR or DQ molecules. It is discovered that these T cells display common or shared TCR structural features among different individuals. The main approach is to first determine over-expressed BV gene(s) in rheumatoid synovium by quantitative real-time PCR. The analysis is performed in peripheral blood (PB), synovial fluid (SF) and synovial lesion tissues (ST) of a group of well-defined RA patients and a control group of patients with osteoarthritis (OA). The serial CDR3 length analyses are performed within the regions spanning 5′BV-3′BC (clonality analysis) by immunoscope technique. Individual CDR3 length of multiple peaks within the V-D-J region has been further dissected using BV and BJ specific primers to identify common clonotypes that use the same BV and BJ genes with similar CDR3 length. These clonotypes have been analyzed by DNA cloning and DNA sequencing.

It is discovered that infiltrating T cells derived from synovial lesion tissue of Chinese RA patients displayed marked skewing of BV gene distribution toward BV14, BV16 and, to a lesser extent, BV20, and that the skewed BV14 pattern seen in the synovial membrane did not appear in paired synovial fluid while the over-expression of BV16 appeared in the synovial fluid. The findings suggest selective activation and accumulation of BV14 and BV16 T cells in the synovial compartment of RA. Although the over-expression of BV14 gene has been reported in T cells derived from RA synovial fluid and membrane previously, the discovery of BV16 over-expression in synovial T cells of RA has not been reported previously. Skewed BV17 and other BV genes were not detected in the Chinese RA patient as what was described in Caucasian RA patients (Zagon et al., 1994; Alam et al., 1995; VanderBorght et al., 2000; Jenkins et al., 1993; and Williams et al., 1992). The observations have indicated that over-expression of BV16 and lack of skewed BV17 and some other BV genes may be characteristically associated with Chinese RA patients while BV14 skewing is common to both Caucasian and Chinese patients with RA. Such discrepancies in BV gene skewing may be attributable to both genetic background (e.g. HLA genes) and environmental factors of geographic significance. In this regard, it is important to note that this cohort of Chinese RA patients is preferentially associated with DRB1*0405 (43% patients), which is different from two other genotypes of DR4 (DRB1*0404 and DRB1*0401) closely linked with Caucasian RA patients (Kerlan-Candon et al., 2001; MacGregor et al., 1995; and Fries et al., 2002). The present study further revealed a trend toward correlation between the over-expression of BV16 but not BV14 in synovial T cells and DRB1*0405 in RA patients. However, the sample size was too small to allow valid statistical analysis. In contrast, no correlation was found between the expression level of BV16 and BV14 and the other frequently used DR and DQ genotypes in these RA patients. These findings support the notion that HLA genotypes and racial background of the individual may influence BV skewing of synovial infiltrating T cells in RA and provides an explanation for characteristic over-expression of BV16 in this Chinese RA population as described in the present study.

It is important to address whether the over-expression of BV14 and BV16 of synovial infiltrating T cells results from autoantigen stimulation in the synovium or whether it is driven by superantigens associated with a common infectious agent(s). The clonality analysis of the skewed BV genes may provide an indication. That is, antigenic stimulation of T cells typically results in oligoclonal expansion while superantigen-induced BV gene skewing is associated characteristically with polyclonal expansion, which can be differentiated by immunoscope patterns of the V-D-J junctional region. In this study, the clonality of the over-expressed BV14 and BV16 seems relatively heterogeneous. Some lesion tissues showed highly restricted oligoclonal patterns while others exhibited a polyclonal profile. The findings support a scenario in which, in some cases, the over-expressed BV is probably driven by autoantigen(s). However, in other cases it is difficult to distinguish between the involvement of multiple autoantigens and superantigens as both situations can result in shift in clonality pattern of the V-DJ region toward a polyclonal profile. The possibility exists that in a later or more chronic stage of the disease, clonality of infiltrating T cells might be obscure and more diverse as inflammatory T cells of heterogeneous nature are recruited through non-specific mechanisms involving various chemokines and cytokines produced in the rheumatoid synovium.

The overexpression of BV14 or BV16 can be determined by a real-time PCR method as disclosed in a co-pending application 60/439,096, which is incorporated herein by reference in its entirety. The real-time PCR method uses specific forward and reverse primer sets of oligonucleotide sequences of BV1-BV25 (SEQ ID NOs: 8-57) and BC (SEQ ID NOs: 58 and 59) as shown in Table 1. The above sets of primers amplify different TCRBV genes and TCRBC gene at the same efficiency, thus the original samples can be quantified with accuracy after amplification by PCR.

TABLE 1

Primers specific for 25 TCRBV genes and TCRBC gene
used in real-time PCR analysis

| | | |
|---|---|---|
| Gene | Sequence 5′ → 3′ | Amplicon (bp) |

| | | | | |
|---|---|---|---|---|
| BV1 | AAGCACCTGATCACAGCAACT | (forward) | (SEQ ID NO. 8) | 209 |
| | TAGTTCAGAGTGCAAGTCAGG | (reverse) | (SEQ ID NO. 9) | |
| BV2 | GGTTATCTGTAAGAGTGGAACCT | (SEQ ID NO. 10) | | 229 |
| | AGGATGGGCACTGGTCACTGT | (SEQ ID NO. 11) | | |
| BV3 | TCGAGATATCTAGTCAAAAGGACG | (SEQ ID NO. 12) | | 228 |
| | GGTGCTGGCGGACTCCAGAAT | (SEQ ID NO. 13) | | |
| BV4 | AAGCAGGGATATCTGTCAACGT | (SEQ ID NO. 14) | | 235 |
| | TTCAGGGCTCATGTTGCTCAC | (SEQ ID NO. 15) | | |
| BV5 | GATCAAAACGAGAGGACAGCA | (SEQ ID NO. 16) | | 217 |
| | AGCACCAAGGCGCTCACATTCA | (SEQ ID NO. 17) | | |
| BV6 | CTCAGGTGTGATCCAATTTCA | (SEQ ID NO. 18) | | 195 |
| | CCCCCGCTCTGTGCGCTGGAT | (SEQ ID NO. 19) | | |
| BV7 | CATGGGAATGACAAATAAGAAGTCT | (SEQ ID NO. 20) | | 214 |
| | TGGCTGCAGGGCGTGTAGGTG | (SEQ ID NO. 21) | | |
| BV8 | CCCCGCCATGAGGTGACAGAG | (SEQ ID NO. 22) | | 239 |
| | GAGTCCCTGGGTTCTGAGGGC | (SEQ ID NO. 23) | | |
| BV9 | CCAAAATACCTGGTCACACAG | (SEQ ID NO. 24) | | 207 |
| | CCAGGGAATTGATGTGAAGATT | (SEQ ID NO. 25) | | |
| BV10 | ACCTAGACTTCTGGTCAAAGCA | (SEQ ID NO. 26) | | 223 |
| | GGACTGGATCTCCAAGGTACA | (SEQ ID NO. 27) | | |
| BV11 | TTATAGGGACAGGAAAGAAGATC | (SEQ ID NO. 28) | | 224 |
| | ATGTGAGGGCCTGGCAGACTC | (SEQ ID NO. 29) | | |
| BV12 | CAAGACACAAGATCACAGAGACA | (SEQ ID NO. 30) | | 224 |
| | GGCAGCAGACTCCAGAGTGAG | (SEQ ID NO. 31) | | |
| BV13 | TGAAGACAGGACAGAGCATGACA | (SEQ ID NO. 32) | | 227 |
| | CACAGATGTCTGGGAGGGAGC | (SEQ ID NO. 33) | | |
| BV14 | ACCCAAGATACCTCATCACAGTG | (SEQ ID NO. 34) | | 242 |
| | AGAGGTCTGGTTGGGGCTGGG | (SEQ ID NO. 35) | | |
| BV15 | TCACAAAGACAGGAAAGAGGATT | (SEQ ID NO. 36) | | 215 |
| | GGGGATGGCAGACTCTAGGGA | (SEQ ID NO. 37) | | |
| BV16 | GTTCCCCAGCCACAGCGTAATA | (SEQ ID NO. 38) | | 235 |
| | CAGTTCTGCAGGCTGCACCTT | (SEQ ID NO. 39) | | |
| BV17 | GTCCCCAAAGTACCTGTTCAGA | (SEQ ID NO. 40) | | 244 |
| | AGCTGTCGGGTTCTTTTGGGC | (SEQ ID NO. 41) | | |
| BV18 | AGACACCTGGTCAGGAGGAGG | (SEQ ID NO. 42) | | 240 |
| | TGCCGAATCTCCTCGCACTAC | (SEQ ID NO. 43) | | |
| BV19 | CCAGGACATTTGGTCAAAGGAAAA | (SEQ ID NO. 44) | | 246 |
| | CAGTGCCGTGTCTCCCGGTTC | (SEQ ID NO. 45) | | |
| BV20 | GACCCTGGTGCAGCCTGTG | (SEQ ID NO. 46) | | 223 |
| | GAGGAGGAGCTTCTTAGAACT | (SEQ ID NO. 47) | | |
| BV21 | CCCAGATATAAGATTACAGAGAAA | (SEQ ID NO. 48) | | 219 |
| | CTGGATCTTGAGAGTGGAGTC | (SEQ ID NO. 49) | | |
| BV22 | CACAGATGGGACAGGAAGTGATC | (SEQ ID NO. 50) | | 221 |
| | GTCCTCCAGCTTTGTGGACCG | (SEQ ID NO. 51) | | |
| BV23 | AAGAGGGAAACAGCCACTCTG | (SEQ ID NO. 52) | | 207 |
| | CAGCTCCAAGGAGCTCATGTT | (SEQ ID NO. 53) | | |
| BV24 | CCAAGATACCAGGTTACCCAGTTT | (SEQ ID NO. 54) | | 228 |
| | CAGGCCTGGTGAGCGGATGTC | (SEQ ID NO. 55) | | |
| BV25 | AAAACATCTTGTCAGAGGGGAA | (SEQ ID NO. 56) | | 238 |
| | TGAATCCTCAAGCTTCGTAGC | (SEQ ID NO. 57) | | |
| TCRBC | CAGCGCCCTTGTGTTGATG | (SEQ ID NO. 58) | | 121 |
| | AAGCGCTGGCAAAAGAAGAA | (SEQ ID NO. 59) | | |

The present invention also identified specific CDR3 sequences and common CDR3 sequence motifs among infiltrating T cells representing over-expressed BV14 and BV16 populations in the rheumatoid synovium. If synovial T cells of BV14 and BV16 are driven by some common autoantigen(s) associated with RA in the context of similar HLA background, the T cell receptor repertoire may be shaped during the course of the disease to develop T cell populations of identical CDR3 or common CDR3 structural features among different RA patients. As the V-D-J region pattern of the over-expressed BV14 and BV16 is relatively diverse, such an attempt is a highly difficult task. The present study first identified similar clonotypes grouped according to common and dominant V-D-J sequence patterns in over-expressed BV14 and BV16. Transcripts containing these common clonotypes were subsequently cloned and analyzed for CDR3 sequences. The study has shown that these common clonotypes have identical V-D-J sequences in synovial lesions derived from different patients with RA. It is remarkable that two identical CDR3 sequences are detected in RA lesions between different patients. The results are reminiscent of similar findings among T cells recognizing myelin basic protein (MBP), a candidate autoantigen for multiple sclerosis (MS). Next, the sequence analysis of over-expressed BV14+ and BV16+ has revealed multiple CDR3 sequence motifs that are shared or common among synovial T cells of different RA patients. Again, the findings support the notion that CDR3 of over-expressed BV14 and BV16 is not random and may result from T cell responses to a common, yet unidentified, autoantigen(s) potentially associated with RA. Since the clonotype analysis by immunoscope was only performed selectively in specimens of >20% expression level of the skewed BV gene(s) and the DNA cloning/sequencing selectively in representative clonotypes, further investigation using sequence-specific primers corresponding to the identified CDR3 sequence may help to evaluate the frequency of the CDR3 structural features in synovial material of a large population of Chinese RA patients.

The present invention is directed to a substantially pure and isolated DNA fragment comprising a nucleic acid sequence as shown in SEQ ID NO. 1 or SEQ ID NO. 2, which is part of the complementary determining region-3 (CDR3) in the Vβ14 family (BV14 gene) and Vβ16 family (BV16 gene) of T cell receptors in patients with rheumatoid arthritis (RA), respectively.

The present invention is also directed to a vaccine which comprises at least one DNA fragment selected from the group consisting of SEQ ID NO. 1 and SEQ ID NO. 2. Preferably, the DNA fragment is present at a concentration range of about 10 μg/ml to about 10 mg/ml.

The present invention is also directed to a substantially pure and isolated peptide having an amino acid sequence selected from the group consisting of SEQ ID NO. 3, SLS, SEQ ID NO. 4, SQD, SLL and SEQ ID NO. 5, which are derived from the CDR3 of T cell receptor beta-chain BV16 (SEQ ID NO. 3 and SLS) or BV14 (SEQ ID NO. 4, SQD, SLL and SEQ ID NO. 5) gene in an individual suffering from rheumatoid arthritis. Also provided is an antibody directed against such peptide.

The present invention is also directed to a vaccine which comprises at least one peptide having an amino acid sequence derived from the CDR3 of a T cell receptor gene selected from the group consisting of BV14 and BV16 in an individual suffering from rheumatoid arthritis.

The present invention is further directed to a method for detecting rheumatoid arthritis. This method advantageously includes obtaining a tissue sample from the suspected individual and a normal individual, respectively; measuring the expression level of BV14 and/or BV16 of T cell receptors in the tissue sample; and comparing the expression level in the suspected and normal individuals. If BV14 and/or BV16 are expressed in a substantially higher level in the suspected individual than in the normal individual, it is indicated that the individual might have rheumatoid arthritis. Preferably, the tissue sample can be obtained from synovial fluid, synovial lesion tissue, or peripheral blood.

The present invention is further directed to a method for detecting rheumatoid arthritis in an individual of Chinese population. This method advantageously includes obtaining a tissue sample from the suspected individual and a normal individual, respectively; measuring the expression level of BV16 of T cell receptors in the tissue sample; and comparing the expression level in the suspected and normal individuals. If BV16 is expressed in a substantially higher level in the suspected individual than in the normal individual, it is indicated that the individual of Chinese population might have rheumatoid arthritis. Preferably, the tissue sample can be obtained from synovial fluid, synovial lesion tissue, or peripheral blood. Since Chinese rheumatoid arthritis patients are found to be preferentially associated with genotype HLA DRB1*0405, this method is especially beneficial for detecting rheumatoid arthritis in an individual of HLA DRB1*0405.

The present invention is further directed to a method for detecting rheumatoid arthritis. This method advantageously includes generating a probe complementary to a DNA fragment having a nucleic acid sequence selected from the group consisting of SEQ ID NO. 1 and SEQ ID NO. 2; obtaining a tissue sample from the suspected individual; and mixing the probe with the tissue sample. In this method a positive hybridization signal indicates a possible detection of rheumatoid arthritis in the suspected individual. Preferably, the tissue sample can be obtained from synovial fluid, synovial lesion tissue, or peripheral blood.

The present invention is still further directed to a method for detecting rheumatoid arthritis. This method advantageously includes generating an antibody directed against a peptide having an amino acid sequence selected from the group consisting of SEQ ID NO. 3, SLS, SEQ ID NO. 4, SQD, SLL and SEQ ID NO. 5; obtaining a tissue sample from the suspected individual; and mixing the antibody with the tissue sample. In this method a positive signal indicates a possible detection of rheumatoid arthritis in the suspected individual. Preferably, the tissue sample can be obtained from synovial fluid, synovial lesion tissue, or peripheral blood.

The present invention is yet further directed to a method for treating rheumatoid arthritis by administering to the individual with an effective amount of an immunogenic T cell receptor peptide to elicit an immune response. Such peptide comprises an amino acid sequence selected from the group consisting of SEQ ID NO. 3, SLS, SEQ ID NO. 4, SQD, SLL and SEQ ID NO. 5.

To enhance the immunogenicity of the peptide, the peptide may be conjugated or immunized with adjuvant.

The present invention is yet further directed to a method for treating rheumatoid arthritis by administering to the individual with an effective amount of an antibody directed against a peptide having an amino acid sequence selected from the group consisting of SEQ ID NO. 3, SLS, SEQ ID NO. 4, SQD, SLL and SEQ ID NO. 5.

The present invention is still yet further directed to a method for treating rheumatoid arthritis. This method advantageously includes administering to the individual with a DNA expression vector comprising a promoter operably linked to a DNA fragment having a nucleic acid sequence encoding a single chain T cell receptor variable beta 16 (Vβ16) peptide, or fragments thereof, and then expressing the DNA fragment in the individual In this method, the DNA fragment is expressed at a level sufficient to elicit an immune response against the encoded peptide thereby preventing onset of rheumatoid arthritis or treating rheumatoid arthritis in the individual.

Preferably, the nucleic acid sequence encodes the complementary determining region-3 (CDR3) of Vβ16 and comprises a sequence as shown in SEQ ID NO. 2. Still preferably, CDR3 of Vβ16 comprises an amino acid sequence selected from the group consisting of SEQ ID NO. 4, SQD, SLL and SEQ ID NO. 5.

The promoter can preferably be inducible or constitutive. Representative examples include β-actin promoter, SV40 early and late promoter, immunoglobulin promoter, human cytomegalovirus promoter, and retroviral LTRs.

Still preferably, the DNA expression vector is administered to the individual subcutaneously, intradermally, intravenously, or orally, and more preferably, to the muscle tissue or spinal fluid of the individual.

The present invention is still yet further directed to a method for treating rheumatoid arthritis. This method advantageously includes administering to the individual with a DNA expression vector comprising a promoter operably linked to a DNA fragment having a nucleic acid sequence encoding a single chain T cell receptor variable beta 14 (Vβ14) peptide, or fragments thereof, and then expressing the DNA fragment in the individual In this method, the nucleic acid sequence comprises a sequence as shown in SEQ ID NO. 1. Upon entering the individual, the DNA fragment is expressed at a level sufficient to elicit an immune response against the encoded peptide thereby preventing onset of rheumatoid arthritis or treating rheumatoid arthritis in the individual.

Preferably, the nucleic acid sequence encodes the complementary determining region-3 (CDR3) of Vβ14, which comprises an amino acid sequence selected from the group consisting of SEQ ID NO. 3 and SLS.

With the recent advent of technology for cloning, genes can be selectively turned off. One possible method of treating rheumatoid arthritis (RA) is to create antisense RNA or DNA molecules that bind specifically with mRNA of a target gene, thereby interrupting the precise molecular mechanism that expresses the gene as a protein. Such antisense technology may be used to treat RA patients by using the above-identified oligonucleotides as shown in SEQ ID NOs. 1 and/or 2, which would be expressed in host cells in an antisense orientation.

The present invention is still yet further directed to a pharmaceutical composition for suppressing pathogenic T cell response in an individual suffering from rheumatoid arthritis. This composition advantageously comprises an immunologically effective amount of a peptide derived from a single chain T cell receptor variable beta 14 (Vβ14) or 16 (Vβ16), or fragments thereof, and a pharmaceutically acceptable carrier. Preferably, the peptide has an amino acid sequence derived from the complementary determining region-3 (CDR3) of Vβ14 or Vβ16 and comprises an amino acid sequence selected from the group consisting of SEQ ID NO. 3, SLS, SEQ ID NO. 4, SQD, SLL and SEQ ID NO. 5.

The following examples are given for the purpose of illustrating various embodiments of the invention and are not meant to limit the present invention in any fashion.

### EXAMPLE 1

Patients and Specimens

A group of 37 Chinese patients with RA as diagnosed according to the American Rheumatism Association criteria were included in this study. Seven patients with osteoarthritis (OA) served as a control group. One of the inclusion criteria was that selected patients had not received steroids or other immunosuppressive treatments 2 months prior to the inclusion. Patients that were on symptomatic treatments were not excluded. Peripheral blood mononuclear cells (PBMC) were prepared from heparinized blood specimens by Ficoll-Hypaque gradient separation. Cells of synovial fluid (SF) were collected from patients of both groups by centrifugation and subsequent washes. Synovial lesion tissues (ST) were obtained by knee synovectomy via anthroscopy from both RA and OA control patients during surgical procedures unrelated to this study. The tissue specimens were cut into small pieces and immediately processed for RNA extraction. The study protocol was approved by the institutional human subjects review board.

### EXAMPLE 2

RNA Extraction Protocol

Total RNA was extracted from peripheral blood (PB), synovial fluid (SF) or synovial tissue (ST) experimental materials (PB, SF and ST samples) using the TRIZOL RNA isolation kit (GIBCOBRL, Carlsbad, Calif.). 50-100 mg of ST was homogenized by DEPC-treated mortar and pestle in 1 ml of TRIZOL reagent. Cells from PB and SF were directly lysed in 1 ml of TRIZOL reagent. Chloroform (0.2 ml) was added in 1 ml of TRIZOL and mixed vigorously. The preparation was centrifuged and mixed with isopropyl alcohol to precipitate RNA according to the manufacturer's protocol.

### EXAMPLE 3

HLA Genotyping

PBMC specimens obtained from all patients were analyzed for HLA DR and DQ genotypes. Briefly, genomic DNA was extracted from EDTA-treated blood of patients and HLA-DRB1 and HLA-DQB1 alleles were determined by PCR with sequence-specific primers (28) using the high resolution SSP UniTray (PEL-FREEZE Clinical System, Brown Deer, Wis.). The primer sets amplifing the alleles were described by the international nomenclature committee of WHO (http://www.anthonynolan.org.uk/HIG/index.html). The panel of HLA-DRB1 alleles and HLA-DQB1 allele were analyzed according to the manufacturer's protocol.

### EXAMPLE 4

Protocols for Determination of BV Gene Usage in Synovial and Blood T Cells by Real-time PCR Analysis

25 TCRBV and TCRBC gene segments were cloned by using TA Cloning® kit (Invitrogen, San Diego, Calif.) and One Shot® TOP10 E.coli competent cells (Invitrogen, San Diego, Calif.) according to the manufacturer's protocol. The oligonucleotide sequences of the BV-specific primers are shown in Table 1. cDNA was synthesized from RNA using random primers and Superscript II (Invitrogen, Carlsbad, Calif.) in a 20-μl reaction. TCR BV gene expression was analyzed by real-time quantitative PCR. An internal reference control for BV-BC amplification and a non-template control containing no cDNA were added to each reaction. Real-time PCR was performed in 96-well optical PCR plates on an ABI 7000 Sequence Detection System (Applied Biosystems, Foster City, Calif.). Briefly, an aliquot of cDNA sample (0.7 μl) was mixed with 25 pairs of BV-specific primers and 1 pair of BC primers (0.1 mM in final solution), respectively, together with SYBR Green PCR Master Mix (Applied Biosystems, Foster City, Calif.) to a final reaction volume of 50 μl. The reaction was performed at 50° C. for 2 min and at 95° for 10 min as hot start activation, which was followed by 40 cycles of reaction at 95° C. for 15 sec and at 60° C. for 1 min. The expression of individual BV genes were calculated based on signal intensity of the PCR reactions according to the following formula:
TCR BVn (%)=[2^{-(BVn CT-BC Ct)}×100/Σ(2^{-(BV1-25-Ct-BC Ct)}×100)]×100.

(Ct refers to threshold cycle).

### EXAMPLE 5

Immunoscope Analysis Method

PCR reactions were performed with 1 μl of cDNA sample derived from ST specimens in the following amplification mixture: 5 μl 10×PCR buffer (100 mM Tris-HCl, pH 8.3 and 500 mM KCl), 3 μl 25 mM magnesium chloride, 1 μl of 10 mM dNTP mix, 0.5 μl of Taq polymerase (5 U/μl) (Invitrogen, Carlsbad, Calif.), 20 pmol of primers (BV14 or BV16 forward primer and BC primer). The PCR amplification profile used was 30 sec at 94° C. for denaturation, 30 sec at 57° C. for annealing, and 30 sec at 72° C. for extension in a total of 40 cycles. Immunoscope analysis was performed with a modified protocol (Even et al., 1995 and Oksenberg 1993). 2 μl of BV14-BC or BV16-BC PCR products were used as templates and run-off reactions were performed with a single internal fluorescent label for each of the 6 FAM (expand)-labeled BC or BJ primers (Table 2). The reaction profile consisted of 30 sec at 94° C. for denaturation temperature and 15 cycles at 94° C. for 45 sec; at 55° C. for 45 sec, at 72° C. for 1 min followed by 72° C. for 5 min as an extension step. The resulting PCR products were then denatured in formamide and analyzed on an Applied Biosystems 3100 Prism using GeneScan 3.7 software (Perkin-Elmer, Boston, Mass.). Labeled products were analyzed separately as one-color electrofluorographs. The relative intensity of signal (RIS) corresponding to CDR3 length was expressed as the area under the experimental peak divided by the area under the control peak found within a Gaussian distribution. Based on the distribution of the signal intensity, specific BJ primers were selected for sequence analysis of CDR3.

TABLE 2
Primers for run-off reactions

| | | |
|---|---|---|
| | | Distance to |
| Primers | Sequence 5′ → 3′ | codon 106, (bp)* |

| | | | |
|---|---|---|---|
| BC (Cb4) | CGA CCT CGG GTG GGA ACA | (SEQ ID NO. 60) | |
| X-BC | X-CAC AGC GAC CTC GGG TGG G | (SEQ ID NO: 61) | 73 |
| X-BJ1.1 | X-ACT GTG AGT CTG GTG CCT TGT | (SEQ ID NO: 62) | 29 |
| X-BJ1.2 | X-ACA ACG GTT AAC TTG GTC CCC GAA | (SEQ ID NO. 63) | 32 |
| X-BJ1.3 | X-GGT CCT CTA CAA CAG TGA GCC AAC | (SEQ ID NO. 64) | 40 |
| X-BJ1.4 | X-AAG AGA GAG AGC TGG GTT CCA CTG | (SEQ ID NO. 65) | 32 |
| X-BJ1.5 | X-GGA GAG TCG AGT TCC ATC A | (SEQ ID NO. 66) | 27 |
| X-BJ1.6 | X-TGT CAC AGT GAG CCT GGT CCC ATT | (SEQ ID NO. 67) | 33 |
| X-BJ2.1 | X-CCT GGC CCG AAG AAC TGC TCA | (SEQ ID NO. 68) | 14 |
| X-BJ2.2 | X-GTC CTC CAG TAC GCT CAG CCT AGA | (SEQ ID NO. 69) | 39 |
| X-BJ2.3 | X-TGC CTG GGC CAA AAT ACT GCG | (SEQ ID NO. 70) | 16 |
| X-BJ2.4 | X-TCC CCG CGC CGA AGT ACT GAA | (SEQ ID NO. 71) | 16 |
| X-BJ2.5 | X-TCG AGC ACC AGG AGC CGC | (SEQ ID NO. 72) | 35 |
| X-BJ2.6 | X-CTG CTG CCG GCC CCG AAA GTC | (SEQ ID NO. 73) | 20 |
| X-BJ2.7 | X-TGA CCG TGA GCC TGG TGC CCG | (SEQ ID NO. 74) | 31 |

X represents 6FAM.
*CDR3 region is defined as within residues 95-106.

### EXAMPLE 6

Protocols for DNA Cloning and Sequencing Analysis of BV14 and BV16 Transcripts

PCR products amplified by either BV14 and BV16 forward primer and BC primer from ST samples were used as templates for second run PCR with specific unlabeled BJ primers (Table 2). The second run PCR products were cloned into the TA cloning vector pCR2.1 (Invitrogen, Carlsbad, Calif.). Fifteen (15) colonies were picked from each sample for colony PCR using BV14 or BV16 forward primer and a corresponding BJ primer. The positive plasmids that showed visible amplification by PCR were selected. Plasmid DNA was prepared from these samples using QIAPrep mini plasmid kit (Qiagen, Valencia, Calif.) and V-DJ region was sequenced with either BV14 or BV16 forward primer to determine the sequence of the CDR3 region.

### EXAMPLE 7

Restricted TCR V Gene Usage in T cells Derived from Synovial Fluid and Lesion Tissues of Patients with RA

A group of well-defined patients with rheumatoid arthritis (RA) and a control group of patients with osteoarthritis (OA) were included in the study as described in Example 1. Clinical characteristics and HLA DR and DQ genotypes are illustrated in Table 3. In this cohort of Chinese RA patients, genotype DRB1*0405 represented the most dominant DR4 (16/37, 43%) compared to two other DR4 genotypes, DRB1*0401 (8%) and DRB1*0404 (3%) that are typically associated with Caucasian RA patients (Kerlan-Candon et al., 2001; MacGregor et al., 1995; and Fries et al., 2002). In addition, DRB1*09012 (35%) and three DQB1 genotypes (0301, 0303 and 0401) were also frequently expressed in this cohort of RA patients (30/-41%).

TABLE 3
Clinical characteristics and HLA genotypes of patients

| | RA | OA | |
|---|---|---|---|
| Total # of patients | 37 | 7 | |
| Age | 56 ± 15 | 64 ± 17 | 5 |
| Sex (M/F) | 11/26 | 2/5 | |
| Disease duration (yrs) | 9 ± 7 | 9 ± 4 | |

| | # of total | % | # of total | % |
|---|---|---|---|---|
| HLA DRB1* | | | | |
| 0405 | 16/37 | 43 | 3/7 | 43 |
| 0401 | 3/37 | 8 | 0/7 | — |
| 0404 | 1/37 | 3 | 0/7 | — |
| 09012 | 13/37 | 35 | 5/7 | 71 |
| 08032 | 6/37 | 16 | 1/7 | 14 |
| 1202 | 7/37 | 19 | 1/7 | 14 |
| 0701 | 5/37 | 14 | 0/7 | — |
| 1501 | 5/37 | 14 | 1/7 | 14 |
| 1302 | 3/37 | 8 | 0/7 | — |
| 1001 | 3/37 | 8 | 0/7 | — |
| 1405 | 2/37 | 5 | 0/7 | — |
| HLA DQB1* | | | | |
| 0303 | 15/37 | 41 | 4/7 | 57 |
| 0301 | 11/37 | 30 | 2/7 | 29 |
| 0401 | 12/37 | 32 | 3/7 | 43 |
| 0601 | 5/37 | 14 | 2/7 | 29 |
| 0201 | 4/37 | 11 | 0/7 | — |
| 0501 | 4/37 | 11 | 0/7 | — |
| 0602 | 4/37 | 11 | 0/7 | — |

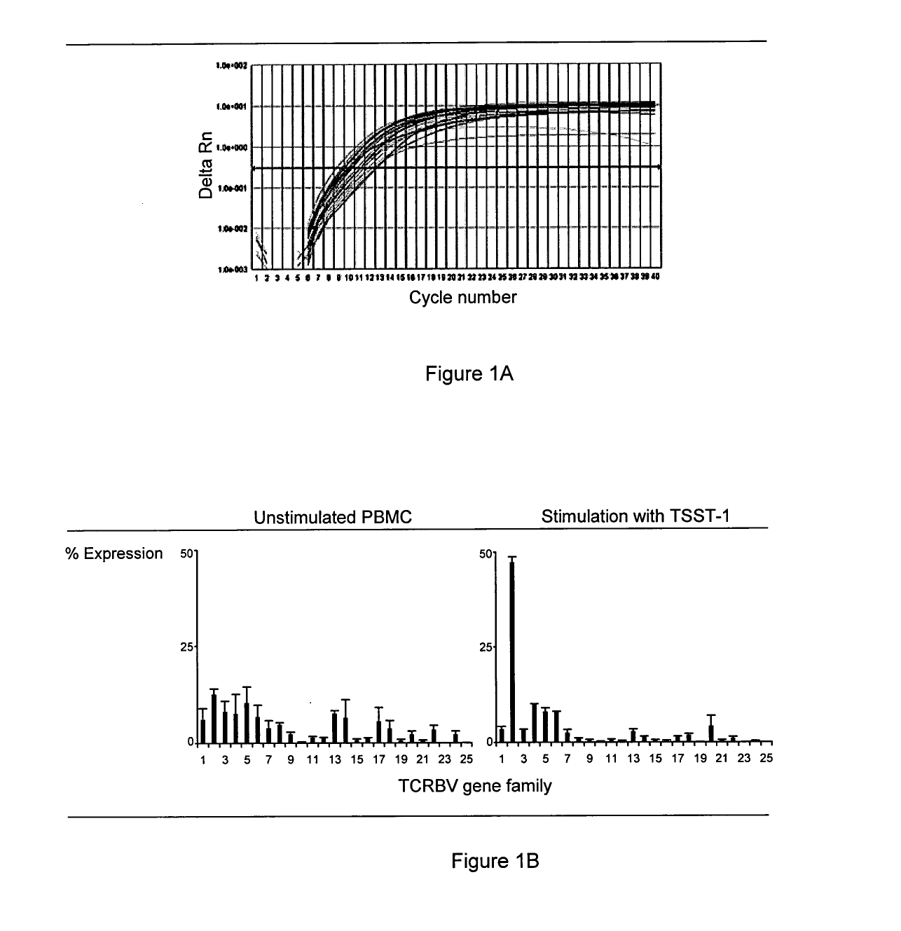

It was first examined whether T cells derived from RA synovial lesions and synovial fluid displayed restricted TCR BV genes and whether the restricted BV genes were associated with HLA genotypes. To this end, PB and synovial (SF and ST) specimens were first analyzed for TCR BV gene usage by real-time quantitative PCR using 25 specific primers. The real-time PCR method used in this study was sensitive and specific for detection of selective expansion of T cells based on BV expression pattern. FIGS. 1A and 1B show the optimization of BV-specific primers for real-time PCR analysis and BV gene analysis of peripheral T cells after stimulation with a superantigen. In FIG. 1A, a set of oligonucleotide primers specific for 25 BV family and BC gene were tested for PCR amplification efficiency profile by an ABI 7000 Sequence Detection System. The results show similar slopes of fluorescence intensity (Delta Rn) in function of cycle numbers, which indicates similar amplification efficiency of TCRBV and TCRBC primers under the PCR conditions described in Example 4. In FIG. 1B, peripheral blood mononuclear cells were prepared separately from four healthy individuals and cultured in the presence and absence of toxic shock syndrome toxin (TSST-1) at the pre-determined concentration of 1 μg/ml for seven days. Cells were collected and washed by centrifugation, and RNA was extracted for real-time PCR analysis using primers specific for 25 BV genes. The PCR conditions are described in above Example 4. Results are presented as mean % expression of the BV genes relative to the BC expression in four cell preparations. As shown in FIG. 1B, selective expansion of BV2 was readily detected by real-time PCR analysis in four peripheral blood mononuclear cell preparations after stimulation with toxic shock syndrome toxin, a superantigen known to activate BV2⁺ T cells.

### EXAMPLE 8

BV Gene Distribution of Synovial and Blood Specimens of RA Patients and Controls by Real-Time PCR Analysis

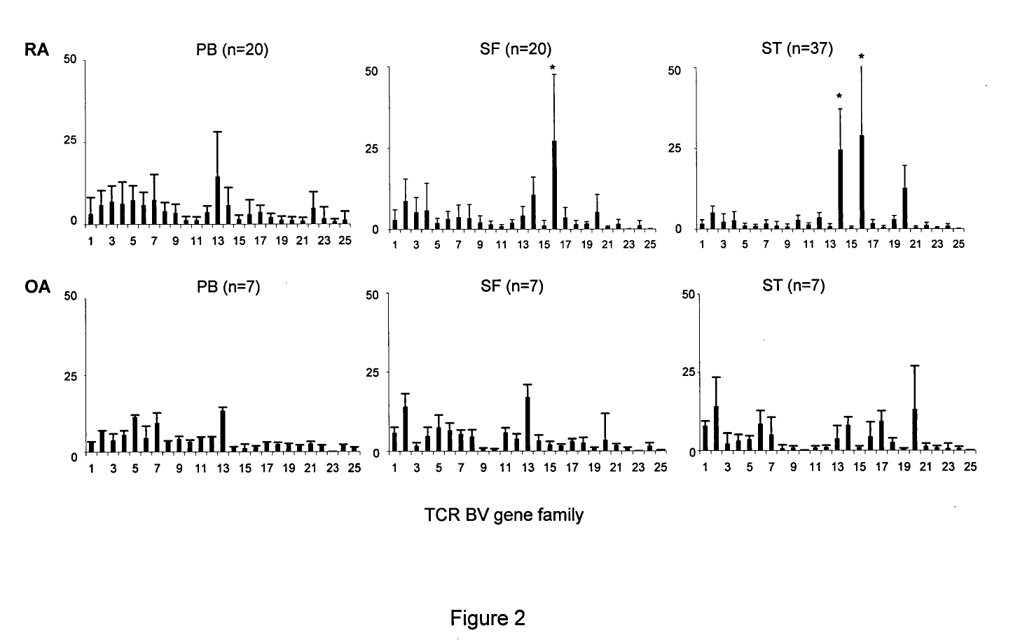

RNA was extracted from 37 ST specimens paired with 20 PB and SF specimens of the same RA patients as some of SF and PB specimens were not available for analysis. BV gene expression in each transcript was analyzed quantitatively by real-time PCR using specific primers for 25 BV genes. Seven all paired specimens of OA patients were tested as a control. BV gene distribution is presented as mean % expression of each BV gene relative to BC expression in Y-axis. Asterisks represent significant differences between the over-expressed BV genes and the remaining BV genes. FIG. 2 reveals a highly significant BV skewing for BV14 (mean expression level of 27%), BV16 (mean expression level of 31%) and, to a lesser extent, BV20 (17%) in RA-derived ST specimens. Similarly, over-expression of BV16 was observed in SF specimens (28%) obtained from the same RA patients while BV14 skewing was not significant in paired SF specimens. In contrast, BV gene distribution appeared highly heterogeneous in RA-derived PB specimens as well as ST and SF specimens obtained from patients with OA. BV14 and BV16 were not over-expressed in these OA-derived synovial specimens. Further analysis indicated a trend for correlation between the over-expression of BV16 but not BV14 with the expression of DRB1*0405 in this group of RA patients. Of the 37 RA patients analyzed for both DR and DQ genotypes and BV gene usage, the expression of BV16 and BV14 was 29% and 11%, respectively, in DRB1*0405 positive individuals (n=16) and 21% and 20% in DRB1*0405 negative individuals (n=21). However, the differences did not reach statistical significance. In contrast, the expression level of both BV14 and BV16 was slightly lower in RA patients of other frequently detected genotypes, including DRB1*09012 and DQB1*0301, 0303 and 0401.

### EXAMPLE 9

Preferential BJ Gene Usage and CDR3 Length Analysis of the Over-Expressed BV14 and BV16 Transcripts of RA Lesion-Derived T Cells

The clonality of the over-expressed BV14 and BV16 of T cells derived from selected synovial material (>20% relative expression level in these specimens) were examined by CDR3 length analysis using immunoscope technique. AsBV14 and BV16 were not over-expressed in ST specimens of OA, two samples (OA2 and OA3) were examined as a control.

Transcripts of over-expressed BV14 derived from synovial material (ST and SF) were analyzed for clonality of the 5′BV-BD-BJ-3′BC region by immunoscope using specific primers for 5′BV14-3′BC. CDR3 length is expressed as peak areas (X-axis). Y-axis represents arbitrary units of fluorescence intensity. Selection of BV14 transcripts for analysis was based on the level of BV expression (>20%) in selected specimens.

Transcripts of over-expressed BV16 derived from synovial material (ST and SF) were analyzed for clonality of the 5′BV-BD-BJ-3′BC region by immunoscope using specific primers for 5′BV16-3′BC. Selection of BV16 transcripts for analysis was based on the level of BV expression (>20%) in selected specimens. BV16 was not expressed in ST specimens of patients RA2, RA17 and RA32.

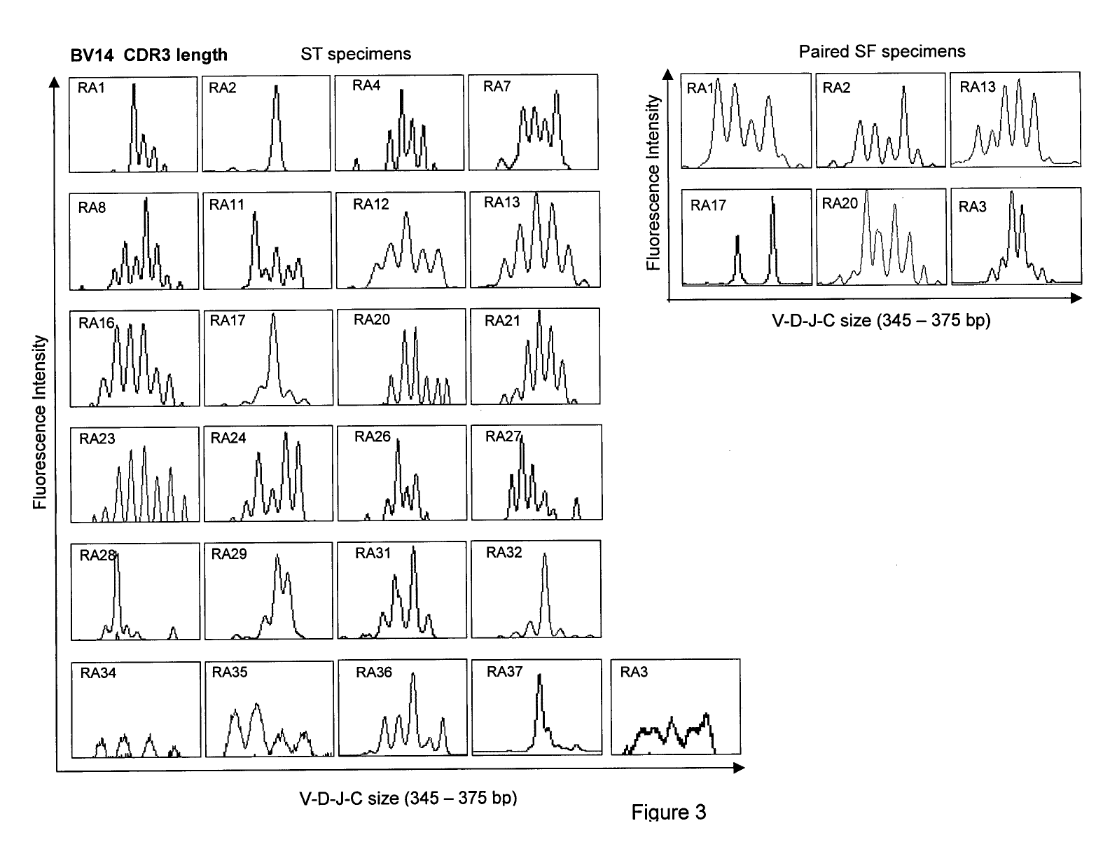

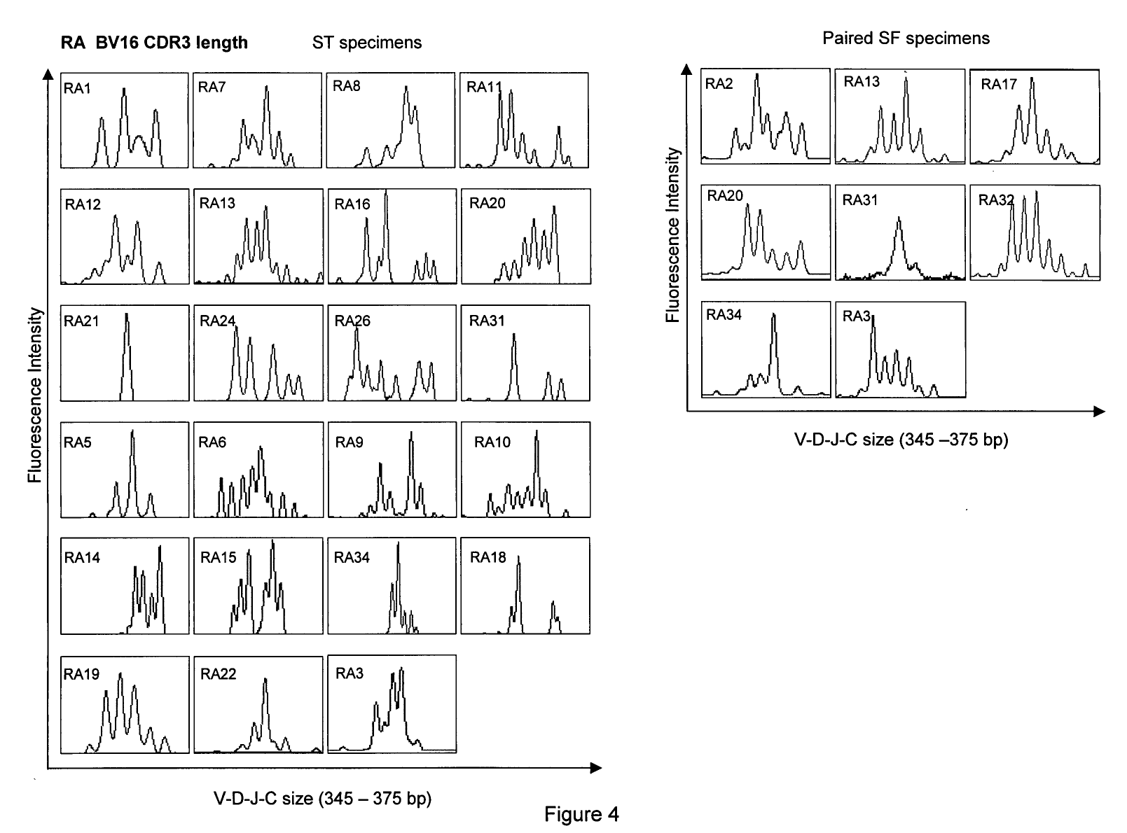

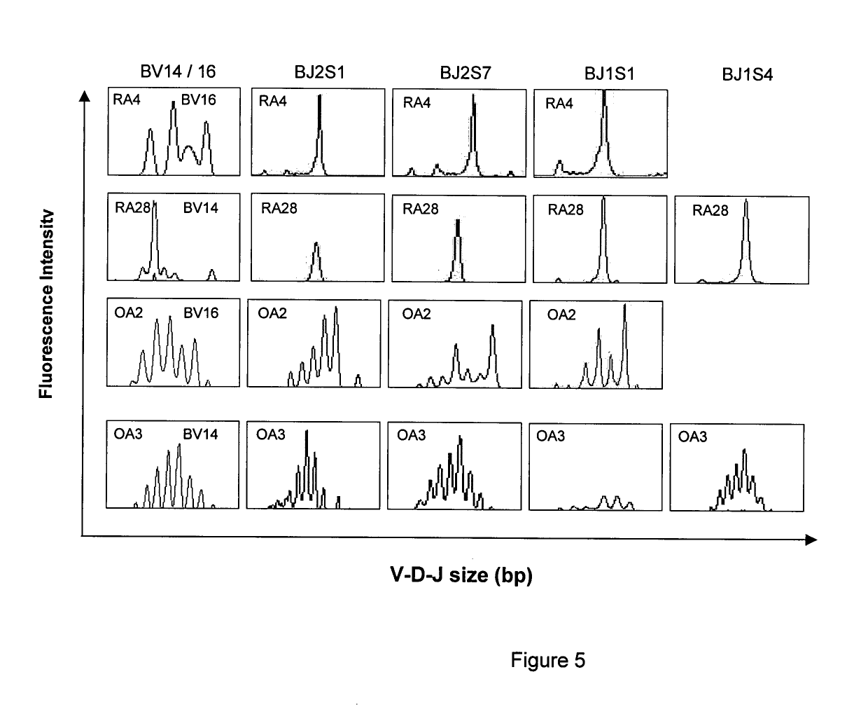

As illustrated in FIGS. 3 and 4, both BV14 and BV16 genes exhibited heterogeneous CDR3 length profile in both SF and ST specimens derived from RA patients when two pairs of 5′BV14-3′BC and 5′BV16-3′BC specific primers were used to analyze the sequence regions between BV14/BV16-BJ-3′BC. Some ST specimens displayed highly limited clonality with characteristic clonotypes (e.g. RA2, RA17, RA28 and RA23 for BV14 and RA21 and RA18 for BV16) while others showed polyclonal patterns. BV14 and BV16 transcripts were further analyzed for CDR3 length profile by immunoscope using BV14 or BV16 primers and a set of primers specific for 13 individual BJ genes, respectively, to identify dominant clonotype patterns in combinations with various BV and BJ. Transcripts derived from ST specimens of two OA patients were included as a control. FIG. 5 shows that diverse patterns of clonality were detected in two control ST specimens of OA patients.

### EXAMPLE 10

Representative Clonotype Patterns Sharing the Same BV and BJ Combinations with Similar CDR3 Length

CDR3 length profile of BV14 and BV16 examined was further dissected and refined by immunoscope using BV14 or BV16 forward primers and reverse primers for 13 BJ genes.

A total of 689 CDR3 length profiles were generated from selected transcripts derived from over-expressed BV14 and/or BV16 genes of ST specimens. The analyses revealed several important findings. First, three to four BJ genes were preferentially used in the context of the over-expressed BV14 and BV16. BJ1S4, BJ2S1 and BJ2S7 were preferentially used with BV16 while BJ1S1, BJ2S1, BJ2S4 and BJ2S7 were associated with BV14. Representative examples are shown in FIG. 5.

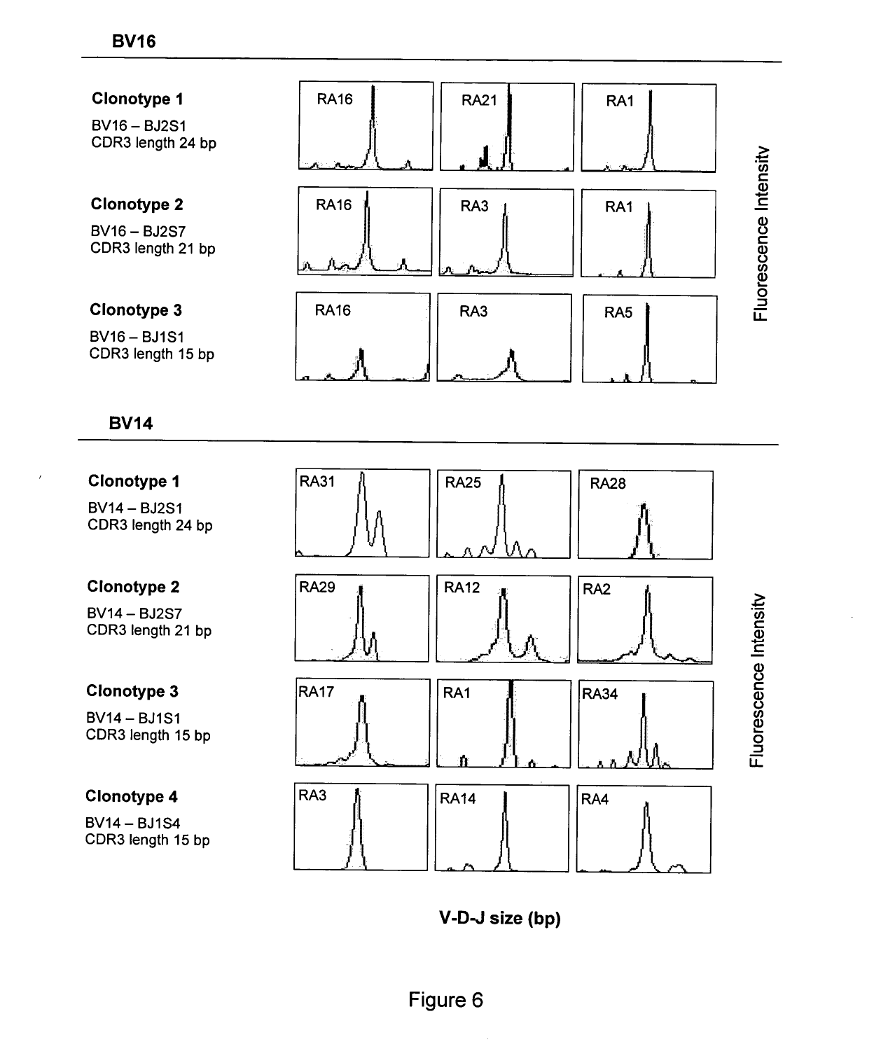

Furthermore, some over-expressed BV14 and BV16 transcripts contained common and dominant clonotypes that had the same structural features of BV and BJ with similar CDR3 length, which were present in various ST specimens of different RA individuals. At least 3 identical clonotypes with CDR3 length of 15, 21 and 24 base pairs, respectively, were detected in BV16 transcripts with BJ2S1, BJ2S7 and BJ1S1 combinations. Similar common clonotypes also appeared in BV14 with BJ2S1, BJ2S7, BJ1S4 and BJ1S1 combinations in ST-derived TCR transcripts of independent RA patients. Representative clonotype patterns are illustrated in FIG. 6, which shows the representative clonotypes that had the same BV and BJ combinations with similar CDR3 length (peak areas, 15, 21 and 24 base pairs for BV16 and BV14). Selected TCR transcripts of common clonotypes were cloned using TA cloning kits and the resulting DNA clones were subsequently analyzed for CDR3 sequences using corresponding BV and BJ primers. The findings raised the possibility that some of these common clonotypes may have identical CDR3 sequences or common CDR3 sequence motifs. Some of the dominant clonotypes were selected for further analysis.

### EXAMPLE 11

CDR3 Sequence Analysis of TCR Transcripts Containing Common Clonotypes

To address whether the identified clonotypes sharing the same V-D-J structural features have the same CDR3 sequence or common CDR3 sequence motifs among different patients, common clonotypes present in TCR transcripts of independent ST specimens are characterized.

Some of the dominant clonotypes were selected for analysis. Selected TCR transcripts of common clonotypes were cloned into TA vector and the resulting DNA clones were subsequently analyzed for CDR3 sequences using corresponding BV and BJ primers. Each cluster of the BV and BJ combination had approximately 15 independent DNA clones randomly selected for sequence analysis. A total of 490 DNA clones were successfully sequenced. The results indicated that most of individual DNA clones of the same cluster had identical CDR3 sequences of the selected clonotypes, indicating in vivo clonal expansion of T cells carrying the clonotypes. The majority of the clonotypes displayed independent CDR3 sequences specific for each individual. Some of the similar clonotypes found in different individuals had identical CDR3 sequences.

Transcripts of over-expressed BV16 containing three similar clonotypes (BV16-2S1/2S7/1S1-CDR3 length 24 bp/21 bp/15 bp) were cloned using TA cloning method. For each DNA cloning, 10-15 resulting DNA clones were sequenced for V-D-J sequence using BV and BJ specific primers.

Transcripts of over-expressed BV14 containing four similar clonotypes (BV14-2S1/2S7/1S4/1S1-CDR3 length 24 bp/21 bp/15 bp) were cloned using TA cloning method. For each DNA cloning, 10-15 resulting DNA clones were sequenced for V-D-J sequence using BV and BJ specific primers.

A CDR3 sequence (SQADGTH, SEQ ID NO. 3) was found in BV16-BJ2S7 transcripts of RA12 and RA16 (Table 4). Another CDR3 sequence (SSGGSLF, SEQ ID NO. 4) appeared in BV14-2S7 transcripts of RA22 and RA23 (Table 5). As shown in Tables 4 and 5, these similar clonotypes exhibited shared/common sequence motifs. Motifs SQD, SLL and SWGG for BV16 were detected in 6/12 BV16 individuals, and the SLS motif was found in 5/14 BV14 individuals. Overall, sequence patterns of SLS, SP- and SS- were found in 86% of all BV14 clonotypes while 77% of BV16 clonotypes had SQ-, SLL and SWGG sequence patterns.

TABLE 4
CDR3 sequences of BV16 clonotypes derived from ST specimens of RA

| Sample | | CDR3 length | |
|---|---|---|---|
| ID | BV-BJ | (basepairs) | V-D-J sequence |
| RA-6 | 16-2S1 | 24 | Y F C A S S Q D S G G G G E Q F F G P G (SEQ ID NO. 75) |
| | | | tatttctgtgccagcagccaagatagcggggggggaggtgagcagttcttcgggcca |
| | | | gga (SEQ ID NO. 76) |
| RA-16 | 16-2S1 | 24 | Y F C A S S R L G Q G Y N E Q F F G P G (SEQ ID NO. 77) |
| | | | Tatttctgtgccagcagccgactgggacagggctacaatgagcagttcttcgggcca |
| | | | gga (SEQ ID NO. 78) |
| RA-21 | 16-2S1 | 24 | Y F C A S S Q D L D S Y N E Q F F G P G (SEQ ID NO. 79) |
| | | | Tatttctgtgccagcagccaagatctggacagctacaatgagcagttcttcgggcca |
| | | | gga (SEQ ID NO. 80) |
| RA-19 | 16-2S1 | 24 | Y F C A S S Q G T S G I T E Q F F G P G (SEQ ID NO. 81) |
| | | | Tatttctgtgccagcagccaggggactagcgggatcactgagcagttcttcgggcca |
| | | | gga (SEQ ID NO. 82) |
| RA-8 | 16-2S1 | 24 | Y F C A S S Q L A G P Y N E Q F F G P G (SEQ ID NO. 83) |
| | | | tatttctgtgccagcagccagctagcgggaccctacaatgagcagttcttcgggccag |
| | | | ga (SEQ ID NO. 84) |
| RA-1 | 16-2S1 | 24 | Y F C A S S L L G T V S Y E Q F F G P G (SEQ ID NO. 85) |
| | | | tatttctgtgccagcagccttctcggcacagtatcctatgagcagttcttcgggccaggc |
| | | | (SEQ ID NO. 86) |
| RA-10 | 16-2S7 | 21 | Y F C A S P L G T A L S Y E Q F F G P G (SEQ ID NO. 87) |
| | | | tatttctgtgccagcccccttgggacagcgctatcctacgagcagtacttcgggccgg |
| | | | gc (SEQ ID NO. 88) |
| RA-12 | 16-2S7 | 21 | Y F C A S S Q A D G T H Y E Q F F G P G (SEQ ID NO. 89) |
| | | | tatttctgtgccagcagccaagctgacgggacccattacgagcagtacttcgggccg |
| | | | ggc (SEQ ID NO. 90) |
| RA-12 | 16-2S7 | 21 | Y F C A S S Q D K G H F Y E Q F F G P G (SEQ ID NO. 91) |
| | | | tatttctgtgccagcagccaagataagggacacttctacgagcagtacttcgggccg |
| | | | ggc (SEQ ID NO. 92) |
| RA-16 | 16-2S7 | 21 | Y F C A S S Q A D G T H Y E Q F F G P G (SEQ ID NO. 93) |
| | | | Tatttctgtgccagcagccaagctgacgggacccattacgagcagtacttcgggccg |
| | | | ggc (SEQ ID NO. 94) |
| RA-14 | 16-2S7 | 21 | Y F C A S S W G G T D I Y E Q F F G P G (SEQ ID NO. 95) |
| | | | Tatttctgtgccagcagctggggcgggacagacatctacgagcagtacttcgggccg |
| | | | ggc (SEQ ID NO. 96) |
| RA-1 | 16-2S7 | 21 | Y F C A S S L L G T V S Y E Q F F G P G (SEQ ID NO. 97) |
| | | | Tatttctgtgccagcagccttctcggcacagtatcctacgagcagtacttcgggccgg |
| | | | gc (SEQ ID NO. 98) |
| RA-17 | 16-1S1 | 15 | Y F C A S S Q G L N T E A F F G Q G (SEQ ID NO. 99) |
| | | | Tatttctgtgccagcagccaaggccttaacactgaagctttctttggacaaggc |
| | | | (SEQ ID NO. 100) |
| RA-5 | 16-1S1 | 15 | Y F C A S R A S R Y T E A F F G Q G (SEQ ID NO. 101) |
| | | | Tatttctgtgccagcagggcaagcaggtacactgaagctttctttggacaaggc |
| | | | (SEQ ID NO. 102) |
| RA-5 | 16-1S1 | 15 | Y F C A S R A S R Y T E A F F G Q G (SEQ ID NO. 103) |
| | | | Tatttctgtgccagcagggcaagcaggtacactgaagctttctttggacaaggc |
| | | | (SEQ ID NO. 104) |
| RA-12 | 16-1S1 | 15 | Y F C A S S T G V N T E A F F G Q G (SEQ ID NO. 105) |
| | | | TatttctgtgccagcAgtacaggggtgaacactgaagctttctttggacaaggc |
| | | | (SEQ ID NO. 106) |
| RA-16 | 16-1S1 | 15 | Y F C A S S L T T N T E A F F G Q G (SEQ ID NO. 107) |
| | | | Tatttctgtgccagcagcctcacaacgaacactgaagctttctttggacaaggc |
| | | | (SEQ ID NO. 108) |
| RA-24 | 16-1S1 | 15 | Y F C A S S Q D S Y T E A F F G Q G (SEQ ID NO. 109) |
| | | | Tatttctgtgccagcagccaagattcgtacactgaagctttctttggacaaggc |
| | | | (SEQ ID NO. 110) |
| RA-1 | 16-1S1 | 15 | Y F C A S S W G G N T E A F F G Q G (SEQ ID NO. 111) |
| | | | Tatttctgtgccagcagctgggggggcaacactgaagctttctttggacaaggc |
| | | | (SEQ ID NO. 112) |

TABLE 5

CDR3 sequences of BV14 clonotypes derived
from ST specimens of RA

| Sample | | CDR3 | |
|---|---|---|---|
| ID | BV-BJ | length (bp) | CDR3 sequence |
| RA-32 | 14-2S1 | 24 | Y F C A S S P T R D R G N E Q F F G P G |
| | | | (SEQ ID NO. 113) |
| | | | tacttctgtgccagcagtcccacgccgggacaggggaaat |
| | | | aatgagcagttcttcgggccagga |
| | | | (SEQ ID NO. 114) |
| RA-13 | 14-2S1 | 24 | Y F C A S S S P I A G S S Y N E Q F F G P G |
| | | | (SEQ ID NO. 115) |
| | | | Tacttctgtgccagcagttccccaatagcggggagctcc |
| | | | aatgagcagttcttcgggccagga |
| | | | (SEQ ID NO. 116) |
| RA-16 | 14-2S1 | 24 | Y F C A S S F W A P T D N E Q F F G P G |
| | | | (SEQ ID NO. 117) |
| | | | Tacttctgtgccagcagtttctgggcccctacgaacaat |
| | | | aatgagcagttcttcgggccagga |
| | | | (SEQ ID NO. 118) |
| RA-23 | 14-2S1 | 24 | Y F C A S S S S S P T S Y N E Q F F G P G |
| | | | (SEQ ID NO. 119) |
| | | | Tacttctgtgccagcagttctagcagccccacctcctac |
| | | | gagcagttcttcgggccagga |
| | | | (SEQ ID NO. 120) |
| RA-27 | 14-2S1 | 24 | Y F C A S S P R E G L L N E Q F F G P G |
| | | | (SEQ ID NO. 121) |
| | | | Tacttctgtgccagcagccctagggagggcctcctcaat |
| | | | aatgagcagttcttcgggccagga |
| | | | (SEQ ID NO. 122) |
| RA-1 | 14-2S1 | 24 | Y F C A S S P W T S G S G N E Q F F G P G |
| | | | (SEQ ID NO. 123) |
| | | | tacttctgtgccagcagtccctggactagcgggagtggt |
| | | | gagcagttcttcgggccagga |
| | | | (SEQ ID NO. 124) |
| RA-32 | 14-2S7 | 21 | Y F C A S S L R T R F Y E Q Y F G P G |
| | | | (SEQ ID NO. 125) |
| | | | Tacttctgtgccagcagtttaaggacacgcttctacgag |
| | | | cagttcttcgggccagga |
| | | | (SEQ ID NO. 126) |
| RA-8 | 14-2S7 | 21 | Y F C A S S L T S G R Q Y E Q Y F G P G |
| | | | (SEQ ID NO. 127) |
| RA-8 | 14-2S7 | 21 | Tacttctgtgccagcagtttgaccagcgggcgtcagtac |
| | | | gagcagttcttcgggccagga |
| | | | (SEQ ID NO. 128) |
| | | | Y F C A S S S G G S L F Y E Q Y F G P G |
| | | | (SEQ ID NO. 129) |
| | | | Tacttctgtgccagcagttccgggggcagtctgttctac |
| | | | gagcagttcttcgggccagga |
| | | | (SEQ ID NO. 130) |
| RA-7 | 14-2S7 | 21 | Y F C A S S L S V G A T Y E Q Y F G P G |
| | | | (SEQ ID NO. 131) |
| RA-7 | 14-2S7 | 21 | Tacttctgtgccagcagtttatcggtcggggctacctac |
| | | | gagcagttcttcgggccagga |
| | | | (SEQ ID NO. 132) |
| | | | Y F C A S S S G G S L F Y E Q Y F G P G |
| | | | (SEQ ID NO. 133) |
| | | | Tacttctgtgccagcagttccgggggcagtctgttctac |
| | | | gagcagttcttcgggccagga |
| | | | (SEQ ID NO. 134) |
| RA-12 | 14-2S7 | 21 | Y F C A S S P S I S S H Y E Q Y F G P G |
| | | | (SEQ ID NO. 135) |
| | | | Tacttctgtgccagcagcccaagtattagttcccactac |
| | | | gagcagttcttcgggccagga |
| | | | (SEQ ID NO. 136) |
| RA-13 | 14-2S7 | 21 | Y F C A S S R D G V S Y E Q Y F G P G |
| | | | (SEQ ID NO. 137) |
| | | | Tacttctgtgccagcagtcgtgatggggtctcctacgag |
| | | | cagttcttcgggccagga |
| | | | (SEQ ID NO. 138) |
| RA-2 | 14-2S7 | 21 | Y F C A S S L S S T G R E Q Y F G P G |
| | | | (SEQ ID NO. 139) |
| | | | Tacttctgtgccagcagtttatcttcgacagggagggag |
| | | | cagtacttcgggccgggc |
| | | | (SEQ ID NO. 140) |
| RA-17 | 14-2S7 | 21 | Y F C A S S L S F R L D Y E Q Y F G P G |
| | | | (SEQ ID NO. 141) |
| | | | Tacttctgtgccagcagtttatcgtttagactagactac |
| | | | gagcagttcttcgggccagga |
| | | | (SEQ ID NO. 142) |
| RA-23 | 14-2S7 | 21 | Y F C A S S P S G Q G S Y E Q Y F G P G |
| | | | (SEQ ID NO. 143) |
| | | | Tacttctgtgccagcagtccgtcgggacaggggtcctac |
| | | | gagcagttcttcgggccagga |
| | | | (SEQ ID NO. 144) |
| RA-1 | 14-2S7 | 21 | Y F C A S S F G T V L S Y E Q Y F G P G |
| | | | (SEQ ID NO. 145) |
| | | | Tacttctgtgccagcagttttgggacagtcctctcctac |
| | | | gagcagttcttcgggccagga |
| | | | (SEQ ID NO. 146) |
| RA-34 | 14-2S7 | 21 | Y F C A S S P R L A G D K E Q Y F G P G |
| | | | (SEQ ID NO. 147) |
| RA-34 | 14-2S7 | 21 | Tacttctgtgccagcagtccccgactagcgggagataaa |
| | | | ggagcagtacttcgggccgggc |
| | | | (SEQ ID NO. 148) |
| | | | Y F C A S S L S A R T T Y E Q Y F G P G |
| | | | (SEQ ID NO. 149) |
| | | | Tacttctgtgccagcagtttaagtgccaggacaacctac |
| | | | gagcagttcttcgggccagga |
| | | | (SEQ ID NO. 150) |
| RA-13 | 14-1S4 | 15 | Y F C A S S L I G G N E K L F L G S G |
| | | | (SEQ ID NO. 151) |
| | | | Tacttctgtgccagcagtttgatagggggcaatgaaaaa |
| | | | ctgttttttggcagtgga |
| | | | (SEQ ID NO. 152) |
| RA-1 | 14-1S4 | 15 | Y F C A S S L S Q E T E A F F G Q G |
| | | | (SEQ ID NO. 153) |
| | | | Tacttctgtgccagagtttatcccaggaaactgaagctt |
| | | | tctttggacaaggc |
| | | | (SEQ ID NO. 154) |
| RA-34 | 14-1S4 | 15 | Y F C A S R A G T G F E K L F F G S G |
| | | | (SEQ ID NO. 155) |
| | | | Tacttctgtgccagcagggccgggacagggtttaaactg |
| | | | ttttttggcagtgga |
| | | | (SEQ ID NO. 156) |
| RA-2 | 14-1S1 | 15 | Y F C A S S L S Q N T E A F F G Q G |
| | | | (SEQ ID NO. 157) |
| | | | Tacttctgtgccagcagtctgtcacagaacactgaagct |
| | | | ttctttggacaaggc |
| | | | (SEQ ID NO. 158) |
| RA-23 | 14-1S1 | 15 | Y F C A S S P R V N T E A F F G Q G |
| | | | (SEQ ID NO. 159) |
| | | | Tacttctgtgccagagtccccgggtcaacactgaagctt |
| | | | tctttggacaaggc |
| | | | (SEQ ID NO. 160) |
| RA-1 | 14-1S1 | 15 | Y F C A S S L S Q E T E A F F G Q G |
| | | | (SEQ ID NO. 161) |
| | | | Tacttctgtgccagagtttatcccaggaaactgaagctt |
| | | | tctttggacaaggc |
| | | | (SEQ ID NO. 162) |
| RA-34 | 14-1S1 | 15 | Y F C A S S L G R N T E A F F G Q G |
| | | | (SEQ ID NO. 163) |
| | | | Tacttctgtgccagcagcctagggaggaacactgaagct |
| | | | ttctttggacaaggc |
| | | | (SEQ ID NO: 164) |
| RA-34 | 14-1S1 | 15 | Y F C A S S S R G Y T E A F F G Q G |
| | | | (SEQ ID NO. 165) |
| RA-34 | 14-1S1 | 15 | Tacttctgtgccagcagttccaggggatacactgaagct |
| | | | ttctttggacaaggc |
| | | | (SEQ ID NO. 166) |
| | | | Y F C A S S S L A T A E A F F G Q G |
| | | | (SEQ ID NO. 167) |
| | | | Tacttctgtgccagcagttccctcgctactgctgaagc |
| | | | tttctttggacaaggc |
| | | | (SEQ ID NO: 168) |

The V-D-J junctional region sequence is expressed in bold. Shared V-D-J sequence (SSGGSLF) and sequence motif (SLS) are underlined.

Any patents or publications mentioned in this specification are indicative of the levels of those skilled in the art to which the invention pertains. Although the invention has been described with reference to specific embodiments, it is not thus limited but is susceptible to various changes and modifications without departing from the spirit thereof.

1. A substantially pure and isolated DNA fragment comprising a nucleic acid sequence as shown in SEQ ID NO. 1.

2. The DNA fragment of claim 1, wherein the DNA fragment comprises at least part of the complementary determining region-3 (CDR3) of T cell receptor beta-chain BV14 gene in an individual suffering from rheumatoid arthritis.

3. A substantially pure and isolated DNA fragment comprising a nucleic acid sequence as shown in SEQ ID NO. 2.

4. The DNA fragment of claim 3, wherein the DNA fragment comprises at least part of the complementary determining region-3 (CDR3) of T cell receptor beta-chain BV16 gene in an individual suffering from rheumatoid arthritis.

5. A vaccine comprising at least one DNA fragment selected from the group consisting of SEQ ID NO. 1 and SEQ ID NO. 2.

6. The vaccine of claim 5, wherein the DNA fragment is present at a concentration range of about 10 μg/ml to about 10 mg/ml.

7. A substantially pure and isolated peptide having an amino acid sequence selected from the group consisting of SEQ ID NO. 3 and SLS.

8. The peptide of claim 7, wherein the peptide has an amino acid sequence derived from the complementary determining region-3 (CDR3) of T cell receptor beta-chain BV14 gene in an individual suffering from rheumatoid arthritis.

9. An antibody directed against the peptide of claim 8.

10. A substantially pure and isolated peptide having an amino acid sequence selected from the group consisting of SEQ ID NO. 4, SQD, SLL and SEQ ID NO. 5.

11. The peptide of claim 10, wherein the peptide has an amino acid sequence derived from the complementary determining region-3 (CDR3) of T cell receptor beta-chain BV16 gene in an individual suffering from rheumatoid arthritis.

12. An antibody directed against the peptide of claim 11.

13. A vaccine comprising at least one peptide having an amino acid sequence derived from the complementary determining region-3 (CDR3) of a T cell receptor gene selected from the group consisting of BV14 and BV16 in an individual suffering from rheumatoid arthritis.

14. The vaccine of claim 13 comprising at least one peptide having an amino acid sequence selected from the group consisting of SEQ ID NO. 3, SLS, SEQ ID NO. 4, SQD, SLL and SEQ ID NO. 5.

15. A method for detecting rheumatoid arthritis in an individual suspected of having rheumatoid arthritis, comprising the steps of:
(a) obtaining a tissue sample from the suspected individual;
(b) measuring the expression level of BV14 and/or BV16 of T cell receptors in the tissue sample; and
(c) repeating steps (a) through (b) on a normal individual;
wherein a substantially higher expression level of BV14 and/or BV16 in the suspected individual than that in the normal individual indicates a possible detection of rheumatoid arthritis in the suspected individual.

16. The method of claim 15, wherein the tissue sample is obtained from synovial fluid, synovial lesion tissue, or peripheral blood of the suspected and normal individuals.

17. A method for detecting rheumatoid arthritis in an individual of Chinese population who is suspected of having rheumatoid arthritis, comprising the steps of:
(a) obtaining a tissue sample from the suspected individual;
(b) measuring the expression level of BV16 of T cell receptors in the tissue sample; and
(c) repeating steps (a) through (b) on a normal individual of Chinese population;
wherein a substantially higher expression level of BV16 in the suspected individual than that in the normal individual indicates a possible detection of rheumatoid arthritis in the suspected individual of Chinese population.

18. The method of claim 17, wherein the tissue sample is obtained from synovial fluid, synovial lesion tissue, or peripheral blood of the suspected and normal individuals of Chinese population.

19. A method for detecting rheumatoid arthritis in an individual suspected of having rheumatoid arthritis, comprising the steps of:
(a) generating a probe complementary to a DNA fragment having a nucleic acid sequence selected from the group consisting of SEQ ID NO. 1 and SEQ ID NO. 2;
(b) obtaining a tissue sample from the suspected individual; and
(c) mixing the probe with the tissue sample, wherein a positive hybridization signal indicates a possible detection of rheumatoid arthritis in the suspected individual.

20. The method of claim 19, wherein the tissue sample is obtained from synovial fluid, synovial lesion tissue, or peripheral blood of the suspected and normal individuals.

21. A method for detecting rheumatoid arthritis in an individual suspected of having rheumatoid arthritis, comprising the steps of:
(a) generating an antibody directed against a peptide having an amino acid sequence selected from the group consisting of SEQ ID NO. 3, SLS, SEQ ID NO. 4, SQD, SLL and SEQ ID NO. 5;
(b) obtaining a tissue sample from the suspected individual; and
(c) mixing the antibody with the tissue sample, wherein a positive signal indicates a possible detection of rheumatoid arthritis in the suspected individual.

22. The method of claim 21, wherein the tissue sample is obtained from synovial fluid, synovial lesion tissue, or peripheral blood of the suspected and normal individuals.

23. A method for treating rheumatoid arthritis in an individual suffering from rheumatoid arthritis, comprising the step of:
administering to the individual with an effective amount of an immunogenic T cell receptor peptide to elicit an immune response, the peptide comprising an amino acid sequence selected from the group consisting of SEQ ID NO. 3, SLS, SEQ ID NO. 4, SQD, SLL and SEQ ID NO. 5.

24. A method for treating rheumatoid arthritis in an individual suffering from rheumatoid arthritis, comprising the step of:
administering to the individual with an effective amount of an antibody directed against a peptide having an amino acid sequence selected from the group consisting of SEQ ID NO. 3, SLS, SEQ ID NO. 4, SQD, SLL and SEQ ID NO. 5.

25. A method of preventing or treating rheumatoid arthritis in an individual, comprising the steps of:
(a) administering to the individual with a DNA expression vector comprising a promoter operably linked to a DNA fragment having a nucleic acid sequence encoding a single chain T cell receptor variable beta 16 (Vβ16) peptide, or fragments thereof; and
(b) expressing the DNA fragment in the individual, the DNA fragment is expressed at a level sufficient to elicit an immune response against the encoded peptide thereby preventing onset of rheumatoid arthritis or treating rheumatoid arthritis in the individual.

26. The method of claim 25, wherein the nucleic acid sequence encodes the complementary determining region-3 (CDR3) of Vβ16.

27. The method of claim 26, wherein the nucleic acid sequence comprises a sequence as shown in SEQ ID NO. 2.

28. The method of claim 26, wherein CDR3 of Vβ16 comprises an amino acid sequence selected from the group consisting of SEQ ID NO. 4, SQD, SLL and SEQ ID NO. 5.

29. The method of claim 25, wherein the promoter is inducible or constitutive.

30. The method of claim 29, wherein the promoter includes β-actin promoter, SV40 early and late promoter, immunoglobulin promoter, human cytomegalovirus promoter, and retroviral LTRs.

31. The method of claim 25, wherein the DNA expression vector is administered to the individual subcutaneously, intradermally, intravenously, or orally.

32. The method of claim 31, wherein the DNA expression vector is administered to the muscle tissue of the individual.

33. The method of claim 31, wherein the DNA expression vector is administered to the spinal fluid of the individual.

34. A method of preventing or treating rheumatoid arthritis in an individual, comprising the steps of:
(a) administering to the individual with a DNA expression vector comprising a promoter operably linked to a DNA fragment having a nucleic acid sequence encoding a single chain T cell receptor variable beta 14 (Vβ14) peptide, or fragments thereof, the nucleic acid sequence comprising a sequence as shown in SEQ ID NO. 1; and
(b) expressing the DNA fragment in the individual, the DNA fragment is expressed at a level sufficient to elicit an immune response against the encoded peptide thereby preventing onset of rheumatoid arthritis or treating rheumatoid arthritis in the individual.

35. The method of claim 34, wherein the nucleic acid sequence encodes the complementary determining region-3 (CDR3) of Vβ14.

36. The method of claim 35, wherein CDR3 of Vβ14 comprises an amino acid sequence selected from the group consisting of SEQ ID NO. 3 and SLS.

37. The method of claim 34, wherein the promoter is inducible or constitutive.

38. The method of claim 37, wherein the promoter includes β-actin promoter, SV40 early and late promoter, immunoglobulin promoter, human cytomegalovirus promoter, and retroviral LTRs.

39. The method of claim 34, wherein the DNA expression vector is administered to the individual subcutaneously, intradermally, intravenously, or orally.

40. The method of claim 39, wherein the DNA expression vector is administered to the muscle tissue of the individual.

41. The method of claim 39, wherein the DNA expression vector is administered to the spinal fluid of the individual.

42. A pharmaceutical composition for suppressing pathogenic T cell response in an individual suffering from rheumatoid arthritis, comprising an immunologically effective amount of a peptide derived from a single chain T cell receptor variable beta 14 (Vβ14) or 16 (Vβ16), or fragments thereof, and a pharmaceutically acceptable carrier.

43. The pharmaceutical composition of claim 42, wherein the peptide has an amino acid sequence derived from the complementary determining region-3 (CDR3) of Vβ14 or Vβ16.

44. The pharmaceutical composition of claim 43, wherein the peptide comprises an amino acid sequence selected from the group consisting of SEQ ID NO. 3, SLS, SEQ ID NO. 4, SQD, SLL and SEQ ID NO. 5.
